SEQUENCE LISTING (112 sequences)

SEQ ID NO: 1 - protein, 12 aa, Artificial Sequence
  Polymethylmethacrylate-binding peptide
IPWWNIRAPLNA

SEQ ID NO: 2 - protein, 12 aa, Artificial Sequence
  Polymethylmethacrylate-binding peptide
TAVMNVVNNQLS

SEQ ID NO: 3 - protein, 12 aa, Artificial Sequence
  Polymethylmethacrylate-binding peptide
VPWWAPSKLSMQ

SEQ ID NO: 4 - protein, 12 aa, Artificial Sequence
  Polymethylmethacrylate-binding peptide
MVMAPHTPRARS

SEQ ID NO: 5 - protein, 12 aa, Artificial Sequence
  Polymethylmethacrylate-binding peptide
TYPNWAHLLSHY

SEQ ID NO: 6 - protein, 7 aa, Artificial Sequence
  Polymethymethacrylate-binding Peptide
TPWWRIT

SEQ ID NO: 7 - protein, 7 aa, Artificial Sequence
  Polymethylmethacrylate-Binding Peptide
DLTLPFH

SEQ ID NO: 8 - protein, 7 aa, Artificial Sequence
  Polymethylmethacrylate-Binding Peptide
GTSIPAM

SEQ ID NO: 9 - protein, 7 aa, Artificial Sequence
  Polymethylmethacrylate-Binding Peptide
HHKHVVA

SEQ ID NO: 10 - protein, 7 aa, Artificial Sequence
  Polymethylmethacrylate-Binding Peptide
HHHKHFM

SEQ ID NO: 11 - protein, 7 aa, Artificial Sequence
  Polymethylmethacrylate-Binding Peptide
HHHRHQG

SEQ ID NO: 12 - protein, 7 aa, Artificial Sequence
  Polymethylmethacrylate-Binding Peptide
HHWHAPR

SEQ ID NO: 13 - protein, 14 aa, Artificial Sequence
  Biotinylated Polymethylmethacrylate-Binding Peptide
  MISC_FEATURE (13)..(13): Biontinylated
  MISC_FEATURE (14)..(14): Amidated
IPWWNIRAPLNAKC

SEQ ID NO: 14 - protein, 13 aa, Artificial Sequence
  Amidated Cysteine-Terminated Polymethylmethacrylate-Binding Peptide
  MISC_FEATURE (13)..(13): Amidated
IPWWNIRAPLNAC

SEQ ID NO: 15 - protein, 12 aa, Artificial Sequence
  Polypropylene-Binding Peptide
TSDIKSRSPHHR

SEQ ID NO: 16 - protein, 12 aa, Artificial Sequence
  Polypropylene-Binding Peptide
HTQNMRMYEPWF

SEQ ID NO: 17 - protein, 7 aa, Artificial Sequence
  Polypropylene-Binding Peptide
LPPGSLA

SEQ ID NO: 18 - protein, 12 aa, Artificial Sequence
  Polypropylene-Binding Peptide
MPAVMSSAQVPR

SEQ ID NO: 19 - protein, 12 aa, Artificial Sequence
  Polypropylene-Binding Peptide
NQSFLPLDFPFR

SEQ ID NO: 20 - protein, 12 aa, Artificial Sequence
  Polypropylene-Binding Peptide
SILSTMSPHGAT

SEQ ID NO: 21 - protein, 12 aa, Artificial Sequence
  Polypropylene-Binding Peptide
SMKYSHSTAPAL

SEQ ID NO: 22 - protein, 12 aa, Artificial Sequence
  Polytertrafluoroethylene-Binding Peptide
ESSYSWSPARLS

SEQ ID NO: 23 - protein, 12 aa, Artificial Sequence
  Polytertrafluoroethylene-Binding Peptide
GPLKLLHAWWQP

SEQ ID NO: 24 - protein, 7 aa, Artificial Sequence
  Polytertrafluoroethylene-Binding Peptide
NALTRPV

SEQ ID NO: 25 - protein, 7 aa, Artificial Sequence
  Polytertrafluoroethylene-Binding Peptide
SAPSSKN

SEQ ID NO: 26 - protein, 12 aa, Artificial Sequence
  Polytertrafluoroethylene-Binding Peptide
SVSVGMKPSPRP

SEQ ID NO: 27 - protein, 12 aa, Artificial Sequence
  Polytertrafluoroethylene-Binding Peptide
SYYSLPPIFHIP

SEQ ID NO: 28 - protein, 12 aa, Artificial Sequence
  Polytertrafluoroethylene-Binding Peptide
TFTPYSITHALL

SEQ ID NO: 29 - protein, 12 aa, Artificial Sequence
  Polytertrafluoroethylene-Binding Peptide
TMGFTAPRFPHY

SEQ ID NO: 30 - protein, 12 aa, Artificial Sequence
  Polytertrafluoroethylene-Binding Peptide
TNPFPPPPSSPA

SEQ ID NO: 31 - protein, 7 aa, Artificial Sequence
  Nylon-Binding Peptide
KTPPTRP

SEQ ID NO: 32 - protein, 7 aa, Artificial Sequence
  Nylon-Binding Peptide
VINPNLD

SEQ ID NO: 33 - protein, 7 aa, Artificial Sequence
  Nylon-Binding Peptide
KVWIVST

SEQ ID NO: 34 - protein, 7 aa, Artificial Sequence
  Nylon-Binding Peptide
AEPVAML

SEQ ID NO: 35 - protein, 7 aa, Artificial Sequence
  Nylon-Binding Peptide
AELVAML

SEQ ID NO: 36 - protein, 7 aa, Artificial Sequence
  Nylon-Binding Peptide
HSLRLDW

SEQ ID NO: 37 - protein, 12 aa, Artificial Sequence
  Polyethylene-Binding Peptide
HNKSSPLTAALP

SEQ ID NO: 38 - protein, 12 aa, Artificial Sequence
  Polyethylene-Binding Peptide
LPPWKHKTSGVA

SEQ ID NO: 39 - protein, 12 aa, Artificial Sequence
  Polyethylene-Binding Peptide
LPWWLRDSYLLP

SEQ ID NO: 40 - protein, 12 aa, Artificial Sequence
  Polyethylene-Binding Peptide
VPWWKHPPLPVP

SEQ ID NO: 41 - protein, 12 aa, Artificial Sequence
  Polyethylene-Binding Peptide
HHKQWHNHPHHA

SEQ ID NO: 42 - protein, 12 aa, Artificial Sequence
  Polyethylene-Binding Peptide
HIFSSWHQMWHR

SEQ ID NO: 43 - protein, 12 aa, Artificial Sequence
  Polyethylene-Binding Peptide
WPAWKTHPILRM

SEQ ID NO: 44 - protein, 13 aa, Artificial Sequence
  Polystyrene-Binding Peptide
TSTASPTMQSKIR

SEQ ID NO: 45 - skipped

SEQ ID NO: 46 - protein, 12 aa, Artificial Sequence
  Polystyrene-Binding Peptide
SHATPPQGLGPQ

SEQ ID NO: 47 - protein, 12 aa, Artificial Sequence
  Hair-Binding Peptide
RTNAADHPAAVT

SEQ ID NO: 48 - protein, 12 aa, Artificial Sequence
  Hair-Binding Peptide
TPPELLHGDPRS

SEQ ID NO: 49 - protein, 7 aa, Artificial Sequence
  Hair-Binding Peptide
NTSQLST

SEQ ID NO: 50 - protein, 7 aa, Artificial Sequence
  Hair-Binding Peptide
DLTLPFH

SEQ ID NO: 51 - protein, 20 aa, Artificial Sequence
  Hair-Binding Peptide
THSTHNHGSPRHTNADAGNP

SEQ ID NO: 52 - protein, 15 aa, Artificial Sequence
  Hair-Binding Peptide
STLHKYKSQDPTPHH

SEQ ID NO: 53 - protein, 12 aa, Artificial Sequence
  Skin-Binding Peptide
TPFHSPENAPGS

SEQ ID NO: 54 - protein, 7 aa, Artificial Sequence
  Skin-Binding Peptide
FTQSLPR

SEQ ID NO: 55 - protein, 12 aa, Artificial Sequence
  Skin-Binding Peptide
KQATFPPNPTAY

SEQ ID NO: 56 - protein, 12 aa, Artificial Sequence
  Skin-Binding Peptide
HGHMVSTSQLSI

SEQ ID NO: 57 - protein, 7 aa, Artificial Sequence
  Skin-Binding Peptide
LSPSRMK

SEQ ID NO: 58 - protein, 7 aa, Artificial Sequence
  Empirically-Generated Hair and Skin-Binding Peptide
RLLRLLR

SEQ ID NO: 59 - protein, 12 aa, Artificial Sequence
  Empirically-Generated Hair and Skin-Binding Peptide
KRGRHKRPKRHK

SEQ ID NO: 60 - protein, 12 aa, Artificial Sequence
  Empirically-Generated Hair and Skin-Binding Peptide
HKPRGGRKKALH

SEQ ID NO: 61 - protein, 18 aa, Artificial Sequence
  Empirically-Generated Hair and Skin-Binding Peptide
KPRPPHGKKHRPKHRPKK

SEQ ID NO: 62 - protein, 18 aa, Artificial Sequence
  Empirically-Generated Hair and Skin-Binding Peptide
RGRPKKGHGKRPGHRARK

SEQ ID NO: 63 - protein, 37 aa, Artificial Sequence
  Peptide Spacer
TSTSKASTTTTSSKTTTTSSKTTTTTSKTSTTSSSST

SEQ ID NO: 64 - protein, 22 aa, Artificial Sequence
  Peptide Spacer
GQGGYGGLGSQGAGRGGLGGQG

SEQ ID NO: 65 - protein, 10 aa, Artificial Sequence
  Peptide Spacer
GPGGYGPGQQ

SEQ ID NO: 66 - protein, 8 aa, Artificial Sequence
  Caspase 3 Cleavage Site.
LESGDEVD

SEQ ID NO: 67 - protein, 63 aa, Artificial Sequence
  Multi-copy Hair-Binding Peptide-Polymer Binding Peptide Conjugate
GSDPGIPWWNIRAPLNAGAGIPWWNIRAPLNAGGSGPGSGGNTSQLSTGGGNTSQLSTGGPKK

SEQ ID NO: 68 - protein, 51 aa, Artificial Sequence
  Multi-copy Hair-Binding Peptide-Polymer Binding Peptide Conjugate
DPRTNAADHPAAVTGGGCGGGIPWWNIRAPLNAGGGCGGGDLTLPFHGGGC

SEQ ID NO: 69 - protein, 49 aa, Artificial Sequence
  Multi-copy Hair-Binding Peptide-Polymer Binding Peptide Conjugate
RTNAADHPAAVTGGGCGGGIPWWNIRAPLNAGGGCGGGDLTLPFHGGGC

SEQ ID NO: 70 - protein, 71 aa, Artificial Sequence
  Triblock peptide-based conjugate
GSDCLEAVAGEPGIPWWNIRAPLNAGAGIPWWNIRAPLNAGGSGPGSGGNTSQLSTGGGNTSQLSTGGPKK

SEQ ID NO: 71 - DNA, 203 bp, Artificial Sequence
  Nucleotide sequence used to prepare triblock peptide-based conjugate
ggatccgaccctggtatcccgtggtggaacattcgcgcacctctgaatgctggtgctggtattccgtggtggaacatccgtgctcctctgaacgcgggtggctccggtccgggctccggtggcaacacgagccaactgagcaccggtggtggcaacacttcccagctgtccaccggcggtccgaaaaagtaataaggcgcgcc

SEQ ID NO: 72 - DNA, 5388 bp, Artificial Sequence
  plasmid pKSIC4-HC77623
agatctcgatcccgcgaaattaatacgactcactatagggagaccacaacggtttccctctagaaataattttgtttaactttaagaaggagatatacatatgcataccccagaacacatcaccgccgtggtacagcgctttgtggctgcgctcaatgccggcgatctggacggcatcgtcgcgctgtttgccgatgacgccacggtggaagagcccgtgggttccgagcccaggtccggtacggctgcgtgtcgtgagttttacgccaactcgctcaaactgcctttggcggtggagctgacgcaggagtgccgcgcggtcgccaacgaagcggccttcgctttcaccgtcagcttcgagtatcagggccgcaagaccgtagttgcgccctgtgatcactttcgcttcaatggcgccggcaaggtggtgagcatccgcgccttgtttggcgagaagaatattcacgcatgccagggatccgatccgactccgccgacgaatgtactgatgctggcaaccaaaggcggtggtacgcattccacgcacaaccatggcagcccgcgccacacgaatgctgacgcaggcaatccgggcggcggcaccccaccaaccaatgtcctgatgctggctactaaaggcggcggcacgcattctacccacaaccatggtagcccgcgccatactaatgcagatgccggcaacccgggcggtggtaccccgccaaccaacgttctgatgctggcgacgaaaggtggcggtacccattccacgcataatcatggcagccctcgccacaccaacgctgatgctggtaatcctggtggcggtaagaagaaataataaggcgcgccgacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 73 - protein, 12 aa, Artificial Sequence
  Hair-binding peptide
EQISGSLVAAPW

SEQ ID NO: 74 - protein, 12 aa, Artificial Sequence
  Hair-binding peptide
TDMQAPTKSYSN

SEQ ID NO: 75 - protein, 12 aa, Artificial Sequence
  Hair-binding peptide
LDTSFPPVPFHA

SEQ ID NO: 76 - protein, 12 aa, Artificial Sequence
  Hair-binding peptide
TPPTNVLMLATK

SEQ ID NO: 77 - protein, 15 aa, Artificial Sequence
  Hair-binding peptide
STLHKYKSQDPTPHH

SEQ ID NO: 78 - protein, 12 aa, Artificial Sequence
  Hair-binding peptide
GMPAMHWIHPFA

SEQ ID NO: 79 - protein, 15 aa, Artificial Sequence
  Hair-binding peptide
HDHKNQKETHQRHAA

SEQ ID NO: 80 - protein, 20 aa, Artificial Sequence
  Hair-binding peptide
HNHMQERYTDPQHSPSVNGL

SEQ ID NO: 81 - protein, 20 aa, Artificial Sequence
  Hair-binding peptide
TAEIQSSKNPNPHPQRSWTN

SEQ ID NO: 82 - protein, 12 aa, Artificial Sequence
  Skin-binding peptide
SVSVGMKPSPRP

SEQ ID NO: 83 - protein, 12 aa, Artificial Sequence
  Skin-binding peptide
TMGFTAPRFPHY

SEQ ID NO: 84 - protein, 12 aa, Artificial Sequence
  Skin-binding peptide
NLQHSVGTSPVW

SEQ ID NO: 85 - protein, 15 aa, Artificial Sequence
  Skin-binding peptide
QLSYHAYPQANHHAP

SEQ ID NO: 86 - protein, 14 aa, Artificial Sequence
  Skin-binding peptide
SGCHLVYDNGFCDH

SEQ ID NO: 87 - protein, 14 aa, Artificial Sequence
  Skin-binding peptide
ASCPSASHADPCAH

SEQ ID NO: 88 - protein, 14 aa, Artificial Sequence
  Skin-binding peptide
NLCDSARDSPRCKV

SEQ ID NO: 89 - protein, 12 aa, Artificial Sequence
  Skin-binding peptide
NHSNWKTAADFL

SEQ ID NO: 90 - protein, 12 aa, Artificial Sequence
  Skin-binding peptide
SDTISRLHVSMT

SEQ ID NO: 91 - protein, 12 aa, Artificial Sequence
  Skin-binding peptide
SPYPSWSTPAGR

SEQ ID NO: 92 - protein, 14 aa, Artificial Sequence
  Skin-binding peptide
DACSGNGHPNNCDR

SEQ ID NO: 93 - protein, 14 aa, Artificial Sequence
  Skin-binding peptide
DWCDTIIPGRTCHG

SEQ ID NO: 94 - protein, 3 aa, Artificial Sequence
  Peptide spacer
GAG

SEQ ID NO: 95 - protein, 9 aa, Artificial Sequence
  Peptide spacer
GGSGPGSGG

SEQ ID NO: 96 - protein, 3 aa, Artificial Sequence
  Peptide spacer
GGG

SEQ ID NO: 97 - protein, 5 aa, Artificial Sequence
  Peptide spacer
GGPKK

SEQ ID NO: 98 - protein, 12 aa, Artificial Sequence
  Shampoo-resistant, PMMA-binding peptide
APWHLSSQYSGT

SEQ ID NO: 99 - protein, 14 aa, Artificial Sequence
  Shampoo-resistant PMMA-binding peptide
GYCLRVDEPTVCSG

SEQ ID NO: 100 - protein, 15 aa, Artificial Sequence
  Shampoo-resistant PMMA-binding peptide
HIHPSDNFPHKNRTH

SEQ ID NO: 101 - protein, 20 aa, Artificial Sequence
  Shampoo-resistant PMMA-binding peptide
HTHHDTHKPWPTDDHRNSSV

SEQ ID NO: 102 - protein, 20 aa, Artificial Sequence
  Shampoo-resistant PMMA-binding peptide
PEDRPSRTNALHHNAHHHNA

SEQ ID NO: 103 - protein, 15 aa, Artificial Sequence
  Shampoo-resistant PMMA-binding peptide
TPHNHATTNHHAGKK

SEQ ID NO: 104 - protein, 15 aa, Artificial Sequence
  Shampoo-resistant PMMA-binding peptide
EMVKDSNQRNTRISS

SEQ ID NO: 105 - protein, 12 aa, Artificial Sequence
  Shampoo-resistant PMMA-binding peptide
HYSRYNPGPHPL

SEQ ID NO: 106 - protein, 12 aa, Artificial Sequence
  Shampoo-resistant PMMA-binding peptide
IDTFYMSTMSHS

SEQ ID NO: 107 - protein, 20 aa, Artificial Sequence
  Shampoo-resistant PMMA-binding peptide
PMKEATHPVPPHKHSETPTA

SEQ ID NO: 108 - protein, 12 aa, Artificial Sequence
  Shampoo-resistant PMMA-binding peptide
YQTSSPAKQSVG

SEQ ID NO: 109 - protein, 15 aa, Artificial Sequence
  Shampoo-resistant PMMA-binding peptide
HLPSYQITQTHAQYR

SEQ ID NO: 110 - protein, 20 aa, Artificial Sequence
  Shampoo-resistant PMMA-binding peptide
TTPKTTYHQSRAPVTAMSEV

SEQ ID NO: 111 - protein, 15 aa, Artificial Sequence
  Shampoo-resistant PMMA-binding peptide
DRIHHKSHHVTTNHF

SEQ ID NO: 112 - protein, 12 aa, Artificial Sequence
  Shampoo-resistant PMMA-binding peptide
WAPEKDYMQLMK

(19) United States
(12) Patent Application Publication
(10) Pub. No.: US 2007/0065387 A1
(43) Pub. Date: Mar. 22, 2007

(54) Method for enhancing the effect of particulate benefit agents

(76) Inventors: William A. Beck, Middletown, DE (US); John P. O'Brien, Oxford, PA (US); Hong Wang, Kennett Square, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/514,804

(22) Filed: Sep. 1, 2006

Related U.S. Application Data
(60) Provisional application No. 60/718,035, filed on Sep. 16, 2005.

(51) Int. Cl. A61K 8/73; A61K 8/64

(52) U.S. Cl. 424/70.13; 424/401; 977/926; 424/70.14

(57) ABSTRACT

A method for applying a particulate benefit agent to a body surface is provided. The method employs a particulate benefit agent coated with a polymer. The polymer-coated benefit agent is applied to a body surface such as hair or skin, in the presence of a composition comprising a peptide having affinity for the polymer. The presence of the polymer-binding peptide in the application serves to extend the binding longevity of the coated particulate benefit agent on the body surface.

### CROSS-REFERENCE TO RELATED APPLICATION

This application claims priority under 35 U.S.C. § 119 from U.S. Provisional Application Ser. No. 60/718,035, filed Sep. 16, 2005.

### FIELD OF THE INVENTION

The invention relates to the use of particulate benefit agents and methods for prolonging the binding of those benefit agents to a body surface. More specifically, the invention provides polymer-coated particle benefit agents in the presence of a composition comprising peptides having affinity for the polymer coating to prolong the binding effect of the benefit agent to body surfaces.

### BACKGROUND OF THE INVENTION

Conditioners and colorants for hair and skin are well-known and frequently used personal care products. The major problem with current conditioners and non-oxidative colorants is that they lack the required durability for long-lasting effects. Oxidative hair dyes provide long-lasting color, but the oxidizing agents they contain cause hair damage. In order to improve the durability of hair and skin care compositions, peptide-based benefit agents, such as hair conditioners and hair colorants, have been developed (Huang et al., copending and commonly owned U.S. Patent Application Publication No. 2005/0050656, and U.S. Patent Application Publication No. 2005/0226839). The peptide-based benefit agents are prepared by coupling a specific peptide sequence that has a high binding affinity to hair or skin with a benefit agent, such as a conditioning or coloring agent. The peptide portion binds to the hair or skin, thereby strongly attaching the benefit agent. These peptide-based benefit agents provide improved durability, but require the coupling of the binding peptide to the benefit agent. Peptide-based sunscreens comprising a skin-binding peptide coupled to an inorganic sunscreen are described by Buseman-Williams et al. in copending and commonly owned U.S. Patent Application Publication No. 2005/0249682.

Peptides with a high binding affinity to hair or skin have been identified using phage display screening techniques (Huang et al., supra; Estell et al., WO 0179479; Murray et al., U.S. Patent Application Publication No. 2002/0098524; Janssen et al., U.S. Patent Application Publication No. 2003/0152976; and Janssen et al., WO 04048399). Additionally, empirically generated hair and skin-binding peptides that are based on positively charged amino acids have been reported (Rothe et al., WO 2004/000257).

Cornwell et al. (U.S. Pat. No. 6,551,361) describe a method for reducing color loss from hair treated with an oxidative hair dye comprising contacting the hair, either before or after treatment of the hair with the oxidative hair dye, with an organic amino compound, such as basic amino acids, urea, guanidine, and salts or mixtures thereof. However, that disclosure does not describe the use of specific polymer-binding peptides, or conjugates comprising polymer-binding peptides coupled to hair or skin-binding peptides to enhance the durability of polymer-coated particulate benefit agents on body surfaces.

Peptides having a binding affinity to polymer and plastic surfaces have been identified using phage display. For example, Adey et al., (Gene 156:27-31 (1995)) describe peptides that bind to polystyrene and polyvinyl chloride surfaces. Additionally, peptides that bind to polyurethane (Murray et al., U.S. Patent Application Publication No. 2002/0098524), polyethylene terephthalate (O'Brien et al., copending and commonly owned U.S. Patent Application Publication No. 2005/0054752), and polystyrene, polyurethane, polycarbonate, and nylon (Grinstaff et al., U.S. Patent Application Publication No. 2003/0185870) have been reported. However, the use of such peptides to enhance the binding of particulate benefit agents to body surfaces has not been described.

The problem to be solved, therefore, is to provide alternative methods to enhance the durability of particulate benefit agents for hair and skin that are simple and easy to implement.

Applicants have addressed the stated problem by discovering that peptides having affinity for a polymer coating on a particulate benefit agent may be used to enhance the durability of the particulate benefit agent on body surfaces. This approach permits the use of one type of polymer binding peptide to be used with many types of polymer-coated particulate benefit agents, thereby eliminating the need for different particle binding peptides for each particle type.

### SUMMARY OF THE INVENTION

A method for enhancing the longevity of the binding of a particulate benefit agent on a body surface is disclosed. Particulate benefit agents coated with a polymer are applied to a body surface in the presence of a polymer binding peptide. The method is particularly suitable for application of pigments, particulate conditioners, and inorganic sunscreens to body surfaces such as hair or skin. Polymer binding peptides may be modified or employed as chimera comprising peptides having affinity for body surfaces such as hair and skin. Polymer coated benefit agents in the presence of polymer binding peptides may be employed in a variety of personal care compositions such as hair colorants and shampoos.

Accordingly in one embodiment the invention provides a method for applying a particulate benefit agent to a body surface comprising:

a) providing a particulate benefit agent coated with a polymer;

b) providing a composition comprising a peptide having affinity for the polymer; and

c) applying the coated particulate benefit agent of (a) with the composition of (b) to a body surface for a time sufficient for the coated benefit agent to bind to the body surface.

In another embodiment the invention provides a personal care composition comprising:

a) a particulate benefit agent coated with a polymer; and

b) a composition comprising a peptide having affinity for the polymer.

In another embodiment the invention provides a diblock, peptide-based conjugate having the general structure [(BSBP)ₘ-(PBP)ₙ]ₓ, wherein
a) BSBP is a body surface binding peptide;
b) PBP is a polymer-binding peptide; and
c) m, n, and x independently range from 1 to about 10.

In an alternate embodiment the invention provides a triblock, peptide-based conjugate having the general structure [[(BSBP)ₘ-S_{q}]ₓ-[(PBP)ₙ-Sᵣ]_{z}]_{y}, wherein
a) BSBP is a body surface binding peptide;
b) PBP is a polymer-binding peptide;
c) S is a molecular spacer; and
d) m, n, x and z independently range from 1 to about 10, y is from 1 to about 5, and where q and r are each independently 0 or 1, provided that both r and q may not be 0.

### BRIEF DESCRIPTION OF FIGURES AND SEQUENCE DESCRIPTIONS

The various embodiments of the invention can be more fully understood from the following detailed description, figure and the accompanying sequence descriptions, which form a part of this application.

FIG. 1 is a plasmid map of the vector pKSIC4-HC77623, described in Example 18.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. § 1.822.

A Sequence Listing is provided herewith on Compact Disk. The contents of the Compact Disk containing the Sequence Listing are hereby incorporated by reference in compliance with 37 CFR 1.52(e). The Compact Disks are submitted in triplicate and are identical to one another. The disks are labeled “Copy 1—Sequence Listing”, “Copy 2 Sequence listing”, and CRF. The disks contain the following file: CL3145 Conv Seq List.ST25 having the following size: 34,000 bytes and which was created Aug. 31, 2006.

SEQ ID NOs:1-14 are the amino acid sequences of polymethylmethacrylate-binding peptides.

SEQ ID NOs:15-21 are the amino acid sequences of polypropylene-binding peptides.

SEQ ID NOs:22-30 are the amino acid sequences of polytetrafluoroethylene-binding peptides.

SEQ ID NOs:31-36 are the amino acid sequences of nylon-binding peptides.

SEQ ID NOs:37-43 are the amino acid sequences of polyethylene-binding peptides.

SEQ ID NOs:44-46 are the amino acid sequences of polystyrene-binding peptides.

SEQ ID NOs:47-52 and 73-81 are the amino acid sequences of hair-binding peptides.

SEQ ID NOs:53-57 and 82-93 are the amino acid sequences of skin-binding peptides.

SEQ ID NOs:58-62 are the amino acid sequences of empirically generated hair and skin-binding peptides.

SEQ ID NOs:63-65, and 94-97 are the amino acid sequences of peptide spacers.

SEQ ID NO:66 is the amino acid sequence of the Caspase 3 cleavage site.

SEQ ID NOs:67-70 are the amino acid sequences of multi-copy hair-binding peptide//polymer binding peptide conjugates.

SEQ ID NO:71 is the nucleotide sequence used to prepare the triblock peptide-based, multi-copy hair-binding peptide//polymer binding peptide conjugate given as SEQ ID NO:70.

SEQ ID NO:72 is the nucleotide sequence of plasmid pKSIC4-HCC77623, which is described in Example 18.

SEQ ID NOs:98-112 are the amino acid sequences of shampoo-resistant polymethylmethacrylate-binding peptides.

### DETAILED DESCRIPTION OF THE INVENTION

The invention relates to a method for enhancing the durability of particulate benefit agents that comprises using a particulate benefit agent coated with a polymer in conjunction with a composition comprising a peptide having affinity for the polymer. The invention is useful because the method may be used to color or condition hair and skin, providing enhanced durability compared to traditional methods.

The following definitions are used herein and should be referred to for interpretation of the claims and the specification.

The term “invention” or “present invention” as used herein is a non-limiting term and is not intended to refer to any single embodiment of the particular invention but encompasses all possible embodiments as described in the specification and the claims.

“PBP” means polymer-binding peptide.

“BSBP” means body surface-binding peptide.

“HBP” means hair-binding peptide.

“SBP” means skin-binding peptide.

“BA” means benefit agent.

The term “peptide” refers to two or more amino acids joined to each other by peptide bonds or modified peptide bonds.

The term “hair-binding peptide” refers to peptide sequences that bind with high affinity to hair. The hair-binding peptides of the invention are from about 7 amino acids to about 50 amino acids, more preferably, from about 7 amino acids to about 25 amino acids, most preferably from about 7 to about 20 amino acids in length.

The term “skin-binding peptide” refers to peptide sequences that bind with high affinity to skin. The skin-binding peptides of the invention are from about 7 amino acids to about 50 amino acids, more preferably, from about 7 amino acids to about 25 amino acids, most preferably from about 7 to about 20 amino acids in length.

The term “polymer-binding peptide” refers to peptide sequences that bind with high affinity to a polymer. The polymer-binding peptides of the invention are from about 7 amino acids to about 50 amino acids, more preferably, from about 7 amino acids to about 25 amino acids, most preferably from about 7 to about 20 amino acids in length.

The term “particulate benefit agent’ is a general term relating to a particulate substance, which when applied to a body surface provides a cosmetic or prophylactic effect. Particulate benefit agents typically include pigments, particulate conditioners, inorganic sunscreens and the like along with other particulate substances commonly used in the personal care industry.

The term “body surface” means any surface of the human body that may serve as a substrate for the application of a particulate benefit agent.

Typical body surfaces include, but are not limited to, hair, skin, nails, teeth, gums, and corneal tissue.

The term “hair” as used herein refers to human hair, eyebrows, and eyelashes.

The term “skin” as used herein refers to human skin, or substitutes for human skin, such as pig skin, Vitro-Skin® and EpiDerm™. Skin as used herein as a body surface will generally comprise a layer of epithelial cells and may additionally comprise a layer of endothelial cells.

The terms “coupling” and “coupled” as used herein refer to any chemical association and include both covalent and non-covalent interactions.

The term “peptide-based conjugate” refers to a composition formed by coupling a body surface-binding peptide with a polymer-binding peptide, either directly or through a molecular spacer.

The term “stringency” as it is applied to the selection of the polymer-binding peptides of the present invention, refers to the concentration of the eluting agent used to elute peptides from the polymer. Higher concentrations of the eluting agent provide more stringent conditions.

The terms “binding affinity” or “affinity” refers to the strength of the interaction of a binding peptide with its respective substrate. The binding affinity is defined herein in terms of the MB₅₀ value, determined in an ELISA-based binding assay.

The term “nanoparticles” are herein defined as particles with an average particle diameter of between 1 and 500 nm. Preferably, the average particle diameter of the particles is between about 1 and 200 nm. As used herein, “particle size” and “particle diameter” have the same meaning. Nanoparticles include, but are not limited to, metallic, semiconductor, polymer, or other organic or inorganic particles, and organic and inorganic pigments.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

The term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

“PCR” or “polymerase chain reaction” is a technique used for the amplification of specific DNA segments (U.S. Pat. Nos. 4,683,195 and 4,800,159).

Standard recombinant DNA and molecular cloning techniques used herein are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley-Interscience (1987).

The invention provides a method for enhancing the durability of particulate benefit agents, such as pigments, particulate conditioners, and inorganic sunscreens, on body surfaces, such as hair and skin, comprising applying to the body surface a polymer-coated particulate benefit agent in conjunction with a composition comprising a peptide having affinity for the polymer coating. The peptides having affinity for the polymer coating, herein also referred to as “polymer-binding peptides”, may be identified using combinatorial methods, such as phage display. Additionally, the composition comprising a peptide having affinity for the polymer coating may further comprise a body surface-binding peptide, such as a hair or skin-binding peptide, either as a free peptide or as a conjugate comprising the polymer-binding peptide coupled to the body surface-binding peptide. In the method of the invention, the composition comprising a peptide having affinity for the polymer coating may be applied concomitantly with the application of the polymer-coated particulate benefit agent, before the application of the benefit agent, or after the application of the benefit agent to seal the benefit agent to the body surface.

Particulate Benefit Agents

The method of the invention may be used in conjunction with a wide variety of particulate benefit agents known in the art of personal care. Examples of particulate benefit agents include, but are not limited to, pigments, particulate conditioning agents, and inorganic sunscreens.

As used herein, the term “pigment” means an insoluble colorant. A wide variety of organic and inorganic pigments alone or in combination may be used in the present invention. Pigments for coloring hair and skin are well known in the art (see for example Green et al. (WO 0107009), incorporated herein by reference, CFTA Intermational Color Handbook, 2^{nd} ed., Micelle Press, England (1992) and Cosmetic Handbook, U.S. Food and Drug Administration, FDA/IAS Booklet (1992)), and are available commercially from various sources (for example Bayer, Pittsburgh, Pa.; Ciba-Geigy, Tarrytown, N.Y.; ICI, Bridgewater, N.J.; Sandoz, Vienna, Austria; BASF, Mount Olive, N.J.; and Hoechst, Frankfurt, Germany). Exemplary pigments include, but are not limited to, D&C Red No. 36, D&C Red No. 30, D&C Orange No. 17, Green 3 Lake, Ext. Yellow 7 Lake, Orange 4 Lake, and Red 28 Lake; the calcium lakes of D&C Red Nos. 7, 11, 31 and 34, the barium lake of D&C Red No. 12, the strontium lake D&C Red No. 13, the aluminum lakes of FD&C Yellow No. 5, of FD&C Yellow No. 6, of FD&C No. 40, of D&C Red Nos. 21, 22, 27, and 28, of FD&C Blue No. 1, of D&C Orange No. 5, of D&C Yellow No. 10, the zirconium lake of D&C Red No. 33; Cromophthal®) Yellow 131AK (Ciba Specialty Chemicals), Sunfast® Magenta 122 (Sun Chemical) and Sunfast® Blue 15:3 (Sun Chemical), iron oxides, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide, black titanium dioxide, titanium dioxide nanoparticles, zinc oxide, barium oxide, ultramarine blue, bismuth citrate, and white minerals such as hydroxyapatite, and Zircon (zirconium silicate), and carbon black particles.

Pigments, by definition, are substantially insoluble and therefore, are used in dispersed form. The pigment may be dispersed using a dispersant or a self-dispersing pigment may be used. When a dispersant is used to disperse the pigment, the dispersant may be any suitable dispersant known in the art, including, but not limited to, random or structured organic polymeric dispersants, as described below; protein dispersants, such as those described by Brueckmann et al. (U.S. Pat. No. 5,124,438); and peptide-based dispersants, such as those described by O'Brien et al (copending and commonly owned U.S. Patent Application Publication No. 2005/0054752). Preferred random organic polymeric dispersants include acrylic polymer and styrene-acrylic polymers. Most preferred are structured dispersants, which include AB, BAB and ABC block copolymers, branched polymers and graft polymers. Preferably the organic polymers comprise monomer units selected from the group consisting of acrylate, methacrylate, butyl methacrylate, 2-ethylhexyl methacrylate, benzyl methacrylate, phenoxyethyl acrylate, ethoxytriethyleneglycol methacrylate, polyethylene glycol methacrylate, polyethylene glycol acrylate, acrylic acid, methacrylic acid, methacrylamide, acrylamide, dimethylaminoethyl methacrylate, hydroxyethyl acrylate, and hydroxyethyl methacrylate, such as those described by Nigan (U.S. Patent Application Publication No. 2004/0232377). Some useful structured polymer dispersants are disclosed in U.S. Pat. No. 5,085,698, EP-A-0556649 and U.S. Pat. No. 5,231,131 (the disclosures of which are incorporated herein by reference). Additionally, pigments may be dispersed using a surface active agent comprising lignin sulfonic acids and a polypeptide, as described by Cioca et al. in U.S. Pat. No. 4,494,994, which is incorporated herein by reference.

The organic polymers on the pigment may optionally be crosslinked by covalent or ionic bonds, generally after they have been applied to the pigment. Crosslinking increases the permanence and environmental-resistance of the polymer coating.

The pigment may optionally be surface-treated prior to coating with organic polymer. Common surface treatments include, but are not limited to, alkyl silane, siloxane, methicone, and dimethicone. Surface treatment increases the range of polymers that have an affinity for the pigment surface.

A self-dispersing pigment is a pigment that has been surface modified with chemically attached, dispersibility imparting groups to allow stable dispersion without a separate dispersant. For dispersion in an aqueous carrier medium, surface modification involves addition of hydrophilic groups and most typically ionizable hydrophilic groups. The self-dispersing pigment may be prepared by grafting a functional group or a molecule containing a functional group onto the surface of the pigment, by physical treatment (such as vacuum plasma), or by chemical treatment (for example, oxidation with ozone, hypochlorous acid or the like). A single type or a plurality of types of hydrophilic functional groups may be bonded to one pigment particle. Self-dispersing pigments are described, for example, in U.S. Pat. No. 5,571,311, U.S. Pat. No. 5,609,671, U.S. Pat. No. 5,968,243, U.S. Pat. No. 5,928,419, U.S. Pat. No. 6,323,257, U.S. Pat. No. 5,554,739, U.S. Pat. No. 5,672,198, U.S. Pat. No. 5,69,8016, U.S. Pat. No. 5,718,746, U.S. Pat. No. 5,749,950, U.S. Pat. No. 5,803,959, U.S. Pat. No. 5,837,045, U.S. Pat. No. 5,846,307, U.S. Pat. No. 5,895,522, U.S. Pat. No. 5,922,118, U.S. Pat. No. 6,123,759, U.S. Pat. No. 6,221,142, U.S. Pat. No. 6,221,143, U.S. Pat. No. 6,281,267, U.S. Pat. No. 6,329,446, U.S. Pat. No. 6,332,919, U.S. Pat. No. 6,375,317, U.S. Pat. No. 6,287,374, U.S. Pat. No. 6,398,858, U.S. 6,402,825, U.S. Pat. No. 6,468,342, U.S. Pat. No. 6,503,311, U.S. Pat. No. 6,506,245, and U.S. Pat. No. 6,852,156. The disclosures of the preceding references are incorporated herein by reference.

Metallic and semiconductor nanoparticles may also be used as hair coloring agents due to their strong emission of light (Vic et al., U.S. Patent Application Publication No. 2004/0010864). The metallic nanoparticles include, but are not limited to, particles of gold, silver, platinum, palladium, iridium, rhodium, osmium, iron, copper, cobalt, and alloys composed of these metals. An “alloy” is herein defined as a homogeneous mixture of two or more metals. The “semiconductor nanoparticles” include, but are not limited to, particles of cadmium selenide, cadmium sulfide, silver sulfide, cadmium sulfide, zinc oxide, zinc sulfide, zinc selenide, lead sulfide, gallium arsenide, silicon, tin oxide, iron oxide, and indium phosphide. The nanoparticles are stabilized and made water-soluble by the use of a suitable organic coating or monolayer. As used herein, monolayer-protected nanoparticles are one type of stabilized nanoparticle. Methods for the preparation of stabilized, water-soluble metal and semiconductor nanoparticles are known in the art, and suitable examples are described by Huang et al. in copending and commonly owned U.S. Patent Application Publication No. 2004/0115345, which is incorporated herein by reference. The color of the nanoparticles depends on the size of the particles. Therefore, by controlling the size of the nanoparticles, different colors may be obtained.

The particulate benefit agent may also be nanoparticles, such as organic nanoparticles; inorganic nanoparticles, such as silica nanoparticles; polymer nanoparticles; and metallic and semiconductor nanoparticles, which serve as hair conditioning agents, specifically, hair straightening aids, hair strengthening aids, and hair volumizing agents.

The particulate benefit agent may also be an inorganic UV sunscreen, which absorbs, reflects, or scatters ultraviolet light at wavelengths from 290 to 400 nanometers. Inorganic UV sunscreen materials are typically inorganic pigments and metal oxides including, but not limited to, titanium dioxide (such as SunSmart available from Cognis Co.), zinc oxide, and iron oxide. A preferred sunscreen is titanium dioxide nanoparticles. Suitable titanium dioxide nanoparticles are described in U.S. Pat. Nos. 5,451,390; 5,672,330; and 5,762,914. Titanium dioxide P25 is an example of a suitable commercial product available from Degussa (Parsippany, N.J.). Other commercial suppliers of titanium dioxide nanoparticles include Kemira (Helsinki, Finland), Sachtleben (Duisburg, Germany) and Tayca (Osaka, Japan).

The titanium dioxide nanoparticles typically have an average particle size diameter of less than 100 nanometers (nm) as determined by dynamic light scattering which measures the particle size distribution of particles in liquid suspension. The particles are typically agglomerates which may range from about 3 nm to about 6000 nm. Any process known in the art can be used to prepare such particles. The process may involve vapor phase oxidation of titanium halides or solution precipitation from soluble titanium complexes, provided that titanium dioxide nanoparticles are produced.

A preferred process to prepare titanium dioxide nanoparticles is by injecting oxygen and titanium halide, preferably titanium tetrachloride, into a high-temperature reaction zone, typically ranging from 400 to 2000° C. Under the high temperature conditions present in the reaction zone, nanoparticles of titanium dioxide are formed having high surface area and a narrow size distribution. The energy source in the reactor may be any heating source such as a plasma torch.

Polymer-Coated Particulate Benefit Agents

For use in the invention, the particulate benefit agent is coated with a polymer coating such that peptides having an affinity for the polymer, identified by combinatorial methods as described below, will bind to the polymer coating. The polymer coating may be formed from many different organic and biological polymers including, but not limited to, polyacrylates, polymethacrylates, polymethylmethacrylates, polycarbonates, polystyrene, polypropylene, polyethylene terephthalate, polyurethanes, polypeptides, lignin, polysaccharides, polyamides, polyimides, polyaramides, and copolymers, (e.g., block and graft copolymers) comprising at least one monomer from methacylates, acrylates or styrene.

If a pigment dispersed with a polymer dispersant, as described above, is used as the particulate benefit agent, the polymer dispersant, may serve as the polymer coating. Any of the polymer dispersants described above may be used. For example, pigments dispersed with a polyacrylate-containing dispersant may be used in conjunction with a polyacrylate-binding peptide. Alternatively, the dispersed pigment may be coated with another polymer as described below.

For pigments and self-dispersing pigments and other particulate benefit agents that are not typically used with a polymer dispersant, the particles may be coated with the polymer using particle coating methods known in the art. Typically, methods used for coating particles are solution-based methods that rely on the application of a polymer coating solution onto the particle surface, followed by the removal of the solvent. For example, the particulate benefit agent may be coated with a polymer by simply mixing the particles with a solution containing the polymer for a time sufficient to coat the particles and then removing the solvent. Additionally, the particulate benefit agents may be coated with a polymer using spray coating techniques, such as those described by Guignon et al. (Drying Technol. 20:419-447 (2002)). Coatings may also be applied with a Wurster coater (see for example, Cardozo et al., U.S. Patent Application Publication No. 2006/0019860). The particulate benefit agents of the invention may also be coated with a polymer using an emulsification-solvent evaporation technique, as described by Rosca et al. (J. Control Release 99:271-280 (2004)). Additionally, particulate benefit agents may be coated with a polymer using the injector mixer method and apparatus described by Schurr (U.S. Pat. No. 4,430,001 and WO 97/007879). In the injector mixer method, small levels of additives are intensely mixed with powders by simultaneously atomizing the coating liquid and dispersing the particles in a gas injector. The method offers the advantages of low water use and very short contact time, which enables the coating of thermally sensitive materials at high temperatures.

Identification of Polymer-Bindinq Peptides

Peptides having affinity for a polymer, also referred to herein as polymer-binding peptides (PBPs), are peptide sequences that bind strongly to a polymer surface. The polymer-binding peptides of the invention are from about 7 amino acids to about 50 amino acids, more preferably, from about 7 amino acids to about 25 amino acids, most preferably from about 7 to about 20 amino acids in length. Suitable polymer-binding peptides may be selected using methods that are well known in the art.

The polymer-binding peptides may be generated randomly and then selected against a specific polymer substrate based upon their binding affinity for the substrate of interest, as described by O'Brien et al. (copending and commonly owned U.S. Patent Application Publication No. 2005/0054752), Adey et al., (Gene 156:27-31, (1995)), Murray et al. (U.S. Patent Application Publication No. 2002/0098524) and Grinstaff et al. (U.S. Patent Application Publication No. 2003/0185870), all of which are incorporated herein by reference. The generation of random libraries of peptides is well known and may be accomplished by a variety of techniques including, bacterial display (Kemp, D. J.; Proc. Natl. Acad. Sci. USA 78(7):4520-4524 (1981), and Helfman et al., Proc. Natl. Acad. Sci. USA 80(1):31-35, (1983)), yeast display (Chien et al., Proc Natl Acad Sci USA 88(21):9578-82 (1991)), combinatorial solid phase peptide synthesis (U.S. Pat. No. 5,449,754, U.S. Pat. No. 5,480,971, U.S. Pat. No. 5,585,275, U.S. Pat. No. 5,639,603), and phage display technology (U.S. Pat. No. 5,223,409, U.S. Pat. No. 5,403,484, U.S. Pat. No. 5,571,698, U.S. Pat. No. 5,837,500). Techniques to generate such biological peptide libraries are well known in the art. Exemplary methods are described in Dani, M., J. of Receptor & Signal Transduction Res., 21(4):447-468 (2001), Sidhu et al., Methods in Enzymology 328:333-363 (2000), Kay et al., Combinatorial Chemistry & High Throughput Screening, Vol. 8:545-551 (2005), and Phage Display of Peptides and Proteins, A Laboratory Manual, Brian K. Kay, Jill Winter, and John McCafferty, eds.; Academic Press, NY, 1996. Additionally, phage display libraries are available commercially from companies such as New England BioLabs (Beverly, Mass.).

A preferred method to randomly generate peptides is by phage display. Phage display is an in vitro selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. In its simplest form, biopanning is carried out by incubating the pool of phage-displayed variants with a target of interest that has been immobilized on a plate or bead, washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

Specifically, the polymer-binding peptides may be selected using the following method. A suitable library of phage-peptides is generated using the methods described above or the library is purchased from a commercial supplier. After the library of phage-peptides has been generated, the library is then contacted with an appropriate amount of the polymer substrate. The library of phage-peptides is dissolved in a suitable solution for contacting the substrate. The test substrate may be suspended in the solution or may be immobilized on a plate or bead. A preferred solution is a buffered aqueous saline solution containing a surfactant. A suitable solution is Tris-buffered saline (TBS) with 0.5% Tween® 20. The solution may additionally be agitated by any means in order to increase the mass transfer rate of the peptides to the polymer substrate, thereby shortening the time required to attain maximum binding.

Upon contact, a number of the randomly generated phage-peptides will bind to the polymer substrate to form a phage-peptide-polymer complex. Unbound phage-peptide may be removed by washing. After all unbound material is removed, phage-peptides having varying degrees of binding affinities for the polymer substrate may be fractionated by selected washings in buffers having varying stringencies. Increasing the stringency of the buffer used increases the required strength of the bond between the phage-peptide and polymer substrate in the phage-peptide-substrate complex.

A number of substances may be used to vary the stringency of the buffer solution in peptide selection including, but not limited to, acidic pH (1.5-3.0); basic pH (10-12.5); high salt concentrations such as MgCl₂ (3-5 M) and LiCI (5-10 M); water; ethylene glycol (25-50%); dioxane (5-20%); thiocyanate (1-5 M); guanidine (2-5 M); urea (2-8 M); and various concentrations of different surfactants such as SDS (sodium dodecyl sulfate), DOC (sodium deoxycholate), Nonidet P40, Triton X-100, Tween® 20, wherein Tween® 20 is preferred. These substances may be prepared in buffer solutions including, but not limited to, Tris-HCl, Tris-buffered saline, Tris-borate, Tris-acetic acid, triethylamine, phosphate buffer, and glycine-HCI, wherein Tris-buffered saline solution is preferred.

It will be appreciated that phage-peptides having increasing binding affinities for the polymer substrate may be eluted by repeating the selection process using buffers with increasing stringencies. The eluted phage-peptides can be identified and sequenced by any means known in the art.

In one embodiment, the following method for generating the polymer-binding peptides of the present invention may be used. A library of combinatorially generated phage-peptides is contacted with the polymer substrate of interest, to form phage peptide-substrate complexes. The phage-peptide-substrate complex is separated from uncomplexed peptides and unbound substrate, and the bound phage-peptides from the phage-peptide-substrate complexes are eluted from the complex, preferably by acid treatment. Then, the eluted phage-peptides are identified and sequenced. To identify peptide sequences that bind to one polymer substrate but not to another, a subtractive panning step may be added. Specifically, the library of combinatorially generated phage-peptides is first contacted with the non-target to remove phage-peptides that bind to it. Then, the non-binding phage-peptides are contacted with the desired polymer substrate and the above process is followed. Alternatively, the library of combinatorially generated phage-peptides may be contacted with the non-target and the desired polymer substrate simultaneously. Then, the phage-peptide-substrate complexes are separated from the phage-peptide-non-target complexes and the method described above is followed for the desired phage-substrate complexes.

Alternatively, a modified phage display screening method for isolating peptides with a higher affinity for polymer substrates may be used. In the modified method, the phage-peptide-substrate complexes are formed as described above. Then, these complexes are treated with an elution buffer. Any of the elution buffers described above may be used. Preferably, the elution buffer is an acidic solution. Then, the remaining, elution-resistant phage-peptide-substrate complexes are used to directly infect/transfect a bacterial host cell, such as E. coli ER2738. The infected host cells are grown in an appropriate growth medium, such as LB (Luria-Bertani) medium, and this culture is spread onto agar, containing a suitable growth medium, such as LB medium with IPTG (isopropyl β-D-thiogalactopyranoside) and S-Gal™. After growth, the plaques are picked for DNA isolation and sequencing to identify the peptide sequences with a high binding affinity for the substrate of interest. Alternatively, PCR may be used to identify the elution-resistant phage-peptides from the modified phage display screening method, described above, by directly carrying out PCR on the phage-peptide-substrate complexes using the appropriate primers, as described by Janssen et al. in U.S. Patent Application Publication No. 2003/0152976, which is incorporated herein by reference.

Additionally, shampoo-resistant polymer-binding peptides may be selected using a modification of the biopanning method for the selection of shampoo-resistant hair-binding peptides, which is described by O'Brien et al. (copending and commonly owned U.S. Patent Application Publication No. 2006/0073111, which is incorporated herein by reference). Similarly, hair conditioner-resistant polymer-binding peptides may be identified using a modification of the method for the selection of hair conditioner-resistant hair-binding peptides, which is described by Wang et al. (copending and commonly owned U.S. patent application Ser. No. 11/359163). The shampoo-resistant and hair conditioner-resistant polymer binding peptides are particularly useful for the treatment of hair because they are able to withstand shampoo or hair conditioner treatment, respectively. In the methods for identifying shampoo-resistant or hair conditioner-resistant polymer-binding peptides, the initial library of phage peptides is dissolved in the matrix of interest (i.e., a shampoo matrix or a hair conditioner matrix) for contacting with the polymer substrate. Alternatively, the phage-peptide-substrate complex, after it is formed by contacting the polymer substrate with the library of phage peptides, as described above, is contacted with the matrix of interest. The contacting of the phage-peptide-substrate complex with the matrix of interest may be repeated one or more times. The biopanning method is then conducted as described above. The shampoo matrix or the hair conditioner matrix may be a full strength commercial product or a dilution thereof. A detailed description of the selection of shampoo-resistant polymethylmethacrylate-binding peptides is given in Example 21.

Suitable examples of polymer-binding peptides, identified using the methods described above, include, but are not limited to, polymethylmethacrylate-binding peptides given as SEQ ID NOs:1-14, shampoo-resistant polymethylmethacrylate-binding peptides given as SEQ ID NOs:98-112, polypropylene-binding peptides given as SEQ ID NOs:15-21, polytetratfluoroethylene-binding peptides given as SEQ ID NOs:22-30, nylon-binding peptides given as SEQ ID NOs: 31-36, polyethylene-binding peptides given as SEQ ID NOs:37-43, and polystyrene-binding peptides given as SEQ ID NOs:44-46. Additionally, polymer-binding peptides known in the art may be used, such as the polystyrene and polyvinyl chloride-binding peptides disclosed by Adey et al. (Gene 156:27-31, (1995)), the polyurethane-binding peptides disclosed by Murray et al. (U.S. Patent Application Publication No. 2002/0098524), the polyethylene terephthalate-binding peptides disclosed by O'Brien et al. (copending and commonly owned U.S. Patent Application Publication No. 2005/0054752), and the polystyrene, polyurethane, polycarbonate, and nylon-binding peptides disclosed by Grinstaff et al., (U.S. Patent Application Publication No. 2003/0185870). It may be desirable to link two or more polymer-binding peptides together, either directly or through a spacer, to enhance the interaction of the peptide with the polymer substrate. Methods to prepare the multiple peptide compositions and suitable spacers are described below.

Production of Polymer-Binding Peptides

The polymer-binding peptides of the present invention may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the polymer-binding peptides of the present invention may be prepared using recombinant DNA and molecular cloning techniques. Genes encoding the polymer-binding peptides may be produced in heterologous host cells, particularly in the cells of microbial hosts, as described by Huang et al. (U.S. Patent Application Publication No. 2005/0050656) and O'Brien et al., supra. The peptides when prepared by recombinant DNA and molecular cloning techniques may further comprise a proline (P) residue at the N-terminus and optionally an aspartic acid (D) residue at the C-terminus. These additional residues result from the use of DP cleavage sites to separate the desired peptide sequence from peptide tags, used to promote inclusion body formation, and between tandem repeats of the peptide sequences (see Example 18).

Body Surface-Binding Peptides

The polymer-binding peptides may be used in combination with body surface-binding peptides including, but not limited to, hair and skin-binding peptides. Body surface-binding peptides (BSBP), as defined herein, are peptide sequences that bind with high affinity to a body surface. Body surface-binding peptides may be generated using combinatorial methods as described above and as described by Huang et al., (copending and commonly owned U.S. Patent Application Publication No. 2005/0050656, and U.S. Patent Application Publication No. 2005/0226839), Estell et al. (WO 0179479); Murray et al., (U.S. Patent Application Publication No. 2002/0098524); Janssen et al., (U.S. Patent Application Publication No. 2003/0152976); and Janssen et al., (WO 04048399). Additionally, shampoo-resistant hair-binding peptides may be selected using a modified biopanning method as described by O'Brien et al. in copending and commonly owned U.S. Patent Application Publication No. 2006/0073111. Similarly, hair conditioner-resistant hair-binding peptides and skin care composition resistant skin-binding peptides may be identified using the methods described by Wang et al. (copending and commonly owned U.S. patent application Ser. No. 11/359,163) and Wang et al. (copending and commonly owned U.S. patent application Ser. No. 11/359,162), respectively. In those methods, either the initial library of phage peptides is dissolved in the matrix of interest (i.e., a shampoo matrix, a hair conditioner matrix, or a skin care composition matrix) for contacting with the substrate, or the phage-peptide substrate complex, after it is formed by contacting the substrate with the library of phage peptides, as described above, is contacted with the matrix of interest. The biopanning method is then conducted as described above. The shampoo matrix, hair conditioner matrix, or skin care composition matrix may be a full strength commercial product or a dilution thereof. Examples of suitable combinatorially generated body surface-binding peptides include, but are not limited to, hair-binding sequences, given as SEQ ID NOs:47-52, and 73-81, and skin-binding sequences, given as SEQ ID NOs:53-57, and 82-93 (see Table A). These body surface-binding peptides may be prepared using the methods described above.

Alternatively, hair and skin-binding peptide sequences may be generated empirically by designing peptides that comprise positively charged amino acids, which can bind to hair and skin via electrostatic interaction, as described by Rothe et al. (WO 2004/000257). The empirically generated hair and skin-binding peptides have between about 7 amino acids to about 50 amino acids, and comprise at least about 40 mole % positively charged amino acids, such as lysine, arginine, and histidine. Peptide sequences containing tripeptide motifs such as HRK, RHK, HKR, RKH, KRH, KHR, HKX, KRX, RKX, HRX, KHX and RHX are most preferred where X can be any natural amino acid but is most preferably selected from neutral side chain amino acids such as glycine, alanine, proline, leucine, isoleucine, valine and phenylalanine. In addition, it should be understood that the peptide sequences must meet other functional requirements in the end use including requirements for solubility, viscosity and compatibility with other components in a formulated product and will therefore vary according to the needs of the application. In some cases the peptide may contain up to 60 mole % of amino acids not comprising histidine, lysine or arginine. Suitable empirically generated hair and skin-binding peptides include, but are not limited to the peptide sequences given as SEQ ID NOs:58-62 (see Table A).

TABLE A

Examples of Hair-Binding and Skin-Binding
Pedtide Sequences

| | | |
|---|---|---|
| | SEQ | |
| | ID | |
| Body Surface | NO: | Sequence |

| | | |
|---|---|---|
| Hair | 47 | RTNAADHPAAVT |
| Hair | 48 | TPPELLHGDPRS |
| (Shampoo Resistant) | | |
| Hair | 49 | NTSQLST |
| (Shampoo Resistant) | | |
| Hair | 50 | DLTLPFH |
| Hair | 51 | THSTHNHGSPRHTNADAGNP |
| Hair | 52 | STLHKYKSQDPTPHH |
| Hair and Skin | 58 | KRGRHKRPKRHK |
| (empirical) | | |
| Hair and Skin | 59 | RLLRLLR |
| (empirical) | | |
| Hair and Skin | 60 | HKPRGGRKKALH |
| (empirical) | | |
| Hair and Skin | 61 | KPRPPHGKKHRPKHRPKK |
| (empirical) | | |
| Hair and Skin | 62 | RGRPKKGHGKRPGHRARK |
| (empirical) | | |
| Hair | 73 | EQISGSLVAAPW |
| Hair | 74 | TDMQAPTKSYSN |
| Hair | 75 | LDTSFPPVPFHA |
| Hair | 76 | TPPTNVLMLATK |
| (Shampoo Resistant) | | |
| Hair | 77 | STLHKYKSQDPTPHH |
| (Conditioner Resistant) | | |
| Hair (Shampoo and | 78 | GMPAMHWIHPFA |
| Conditioner Resistant) | | |
| Hair (Shampoo and | 79 | HDHKNQKETHQRHAA |
| Conditioner Resistant) | | |
| Hair (Shampoo and | 80 | HNHMQERYTDPQHSPSVNGL |
| Conditioner Resistant) | | |
| Hair (Shampoo and | 81 | TAEIQSSKNPNPHPQRSWTN |
| Conditioner Resistant) | | |
| Skin | 53 | TPFHSPENAPGS |
| Skin | 54 | FTQSLPR |
| Skin | 55 | KQATFPPNPTAY |
| Skin | 56 | HGHMVSTSQLSI |
| Skin | 57 | LSPSRMK |
| Skin | 82 | SVSVGMKPSPRP |
| (Body Wash Resistant) | | |
| Skin | 83 | TMGFTAPRFPHY |
| (Body Wash Resistant) | | |
| Skin | 84 | NLQHSVGTSPVW |
| (Body Wash Resistant) | | |
| Skin | 85 | QLSYHAYPQANH HAP |
| (Body Wash Resistant) | | |
| Skin | 86 | SGCHLVYDNGFCDH |
| (Body Wash Resistant) | | |
| Skin | 87 | ASCPSASHADPCAH |
| (Body Wash Resistant) | | |
| Skin | 88 | NLCDSARDSPRCKV |
| (Body Wash Resistant) | | |
| Skin | 89 | NHSNWKTAADFL |
| (Body Wash Resistant) | | |
| Skin | 90 | SDTISRLHVSMT |
| (Body Wash Resistant) | | |
| Skin | 91 | SPYPSWSTPAGR |
| (Body Wash Resistant) | | |
| Skin | 92 | DACSGNGHPNNCDR |
| (Body Wash Resistant) | | |
| Skin | 93 | DWCDTIIPGRTCHG |
| (Body Wash Resistant) | | |

The body surface-binding peptides may be used in combination with the polymer-binding peptides of the invention to enhance the effects of particle-based benefit agents in various ways. The body surface-binding peptide may be added to the composition comprising the peptide having affinity for the polymer, as described below. Alternatively, a conjugate comprising a body surface-binding peptide coupled to a polymer-binding peptide may be used. The coupling interaction may be a covalent bond or a non-covalent interaction, such as hydrogen bonding, electrostatic interaction, hydrophobic interaction, or Van der Waals interaction. In the case of a non-covalent interaction, the peptide-based conjugate may be prepared by mixing the body surface-binding peptide with a polymer-binding peptide and an optional spacer and allowing sufficient time for the interaction to occur. The unbound materials may be separated from the resulting peptide-based conjugate using methods known in the art, for example, chromatographic methods.

The peptide-based conjugates may also be prepared by covalently attaching a specific body surface-binding peptide, for example a hair or a skin-binding peptide, to a polymer-binding peptide, either directly or through a molecular spacer, as described by Huang et al. in U.S. Patent Application Publication No. 2005/0050656. Any suitable known peptide or protein conjugation chemistry may be used to form the peptide-based conjugates of the invention. Conjugation chemistries are well-known in the art (see for example, Hermanson, Bioconjugate Techniques, Academic Press, New York (1996)). Suitable coupling agents include, but are not limited to, carbodiimide coupling agents, acid chlorides, isocyanates, epoxides, maleimides, and other functional coupling reagents that are reactive toward terminal amine and/or carboxylic acid groups, and sulfhydryl groups on the peptides. Additionally, it may be necessary to protect reactive amine or carboxylic acid groups on the peptide to produce the desired structure for the peptide-based conjugate. The use of protecting groups for amino acids, such as t-butyloxycarbonyl (t-Boc), are well known in the art (see for example Stewart et al., supra; Bodanszky, supra; and Pennington et al., supra).

It may also be desirable to couple the body surface-binding peptide to the polymer-binding peptide via a molecular spacer. The spacer serves to separate the peptide sequences to ensure that they do not interfere with the binding of the peptides to the body surface or the polymer-coated benefit agent. The molecular spacer may be any of a variety of molecules, such as alkyl chains, phenyl compounds, ethylene glycol, amides, esters and the like. Preferred spacers are hydrophilic and have a chain length from 1 to about 100 atoms, more preferably, from 2 to about 30 atoms. Examples of preferred spacers include, but are not limited to ethanolamine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, and ethyl, propyl, hexyl, steryl, cetyl, and palmitoyl alkyl chains. The molecular spacer may be covalently attached to the peptides using any of the coupling chemistries described above. In order to facilitate incorporation of the spacer, a bifunctional coupling agent that contains a spacer and reactive groups at both ends for coupling to the peptides may be used. Suitable bifunctional coupling agents are well known in the art and include, but are not limited to diamines, such a as 1,6-diaminohexane; dialdehydes, such as glutaraldehyde; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyanates, such as hexamethylenediisocyanate; bis oxiranes, such as 1,4 butanediyl diglycidyl ether; dicarboxylic acids, such as succinyidisalicylate; and the like. Heterobifunctional coupling agents, which contain a different reactive group at each end, may also be used.

Additionally, the molecular spacer may be a peptide comprising any amino acid and mixtures thereof. The preferred peptide spacers are comprised of the amino acids proline, lysine, glycine, alanine, and serine, and mixtures thereof. The peptide spacer may be from 1 to about 50 amino acids, preferably from 1 to about 20 amino acids in length. Exemplary peptide spacers include, but are not limited, to SEQ ID NOs:63-65, and 94-97. In addition, the peptide spacer may contain a specific enzyme cleavage site, such as the protease Caspase 3 site, given as SEQ ID NO:66, which allows for the enzymatic removal of the particulate benefit agent from the body surface. These peptide spacers may be linked to the binding peptide sequences by any method known in the art. For example, the entire peptide-based conjugate may be prepared using the standard peptide synthesis methods described above. In addition, the binding peptides and peptide spacer blocks may be combined using carbodiimide coupling agents (see for example, Hermanson, Bioconjugate Techniques, Academic Press, New York (1996)), diacid chlorides, diisocyanates and other difunctional coupling reagents that are reactive to terminal amine and/or carboxylic acid groups on the peptides. Altematively, the entire peptide-based conjugate may be prepared using the recombinant DNA and molecular cloning techniques described above. The spacer may also be a combination of a peptide spacer and an organic spacer molecule, which may be prepared using the methods described above.

It may also be desirable to have multiple copies of the body surface-binding peptide and the polymer-binding peptide coupled together to enhance the interaction between the peptide-based conjugate and the polymer-coated benefit agent and the body surface, as described by Huang et al. (U.S. Patent Application Publication No. 2005/0050656). Either multiple copies of the same body surface-binding peptide and polymer-binding peptide or a combination of different body surface-binding peptides and polymer-binding peptides may be used. The multi-copy peptide-based conjugates may comprise various spacers as described above. Exemplary multi-copy body surface-binding peptide//polymer-binding peptide conjugates include, but are not limited to, the multi-copy hair-binding peptide//polymer binding peptide conjugates given as SEQ ID NOs:67-70.

In one embodiment of the invention, the peptide-based conjugate is a diblock composition comprising a body surface-binding peptide (BSBP) and a polymer-binding peptide (PBP), having the general structure [(BSBP)ₘ-(PBP)ₙ]ₓ, where n and m independently range from 1 to about 10, preferably from 1 to about 5, and x may be 1 to about 10. In another embodiment, the peptide-based conjugate comprises a molecular spacer (S) separating the body surface-binding peptide from the polymer-binding peptide, as described above. Multiple copies of the body surface-binding peptide and the polymer-binding peptide may also be used and the multiple copies of the body surface-binding peptide and the polymer-binding peptide may be separated from themselves and from each other by molecular spacers. In this embodiment, the peptide-based conjugate is a triblock composition comprising a body surface-binding peptide, a spacer, and polymer-binding peptide, having the general structure [[(BSBP)ₘ-S_{q}]ₓ-[(PBP)ₙ-Sᵣ]_{z}]_{y}, where m, n, x and z independently range from 1 to about 10, y is from 1 to about 5, and where q and r are each independently 0 or 1, provided that both r and q may not be 0.

In another embodiment, the body surface-binding peptide is a hair-binding peptide and the peptide-based conjugate is a diblock composition comprising the hair-binding peptide (HBP) and a polymer-binding peptide (PBP), having the general structure [(HBP)ₘ-(PBP)ₙ]ₓ where n and m independently range from 1 to about 10, preferably from 1 to about 5, and x may be 1 to about 10.

In another embodiment, the body surface-binding peptide is a hair-binding peptide and the peptide-based conjugate is a triblock composition comprising the hair-binding peptide (HBP), a spacer (S), and a polymer-binding peptide (PBP), having the general structure [[(HBP)ₘ-S_{q}]ₓ-[(PBP)ₙ-Sᵣ]_{z}]_{y}, where m, n, x and z independently range from 1 to about 10, y is from 1 to about 5, and where q and r are each independently 0 or 1, provided that both r and q may not be 0.

In another embodiment, the body surface-binding peptide is a skin-binding peptide and the peptide-based conjugate is a diblock composition comprising the skin-binding peptide (SBP) and a polymer-binding peptide (PBP), having the general structure [(SBP)ₘ-(PBP)ₙ]ₓ, where n and m independently range from 1 to about 10, preferably from 1 to about 5, and x may be 1 to about 10.

In another embodiment, the body surface-binding peptide is a skin-binding peptide and the peptide-based conjugate is a triblock composition comprising the skin-binding peptide (SBP), a spacer (S), and polymer-binding peptide (PBP), having the general structure [[(SBP)ₘ-S_{q}]ₓ-[(PBP)ₙ-Sᵣ]_{z}]_{y}, where m, n, x and z independently range from 1 to about 10, y is from 1 to about 5, and where q and r are each independently 0 or 1, provided that both r and q may not be 0.

It should be understood that as used herein, BSBP, HBP, SBP, and PBP are generic designations and are not meant to refer to a single body surface-binding peptide, hair-binding peptide, skin-binding peptide, or polymer-binding peptide sequence, respectively. Where m, n, x or z as used above, is greater than 1, it is well within the scope of the invention to provide for the situation where a series of body surface-binding peptides (e.g., hair or skin-binding peptides) of different sequences and polymer-binding peptides of different sequences may form a part of the composition. In addition, “S” is also a generic term and is not meant to refer to a single spacer. Where q, x, r, or z, as used above, is greater than 1, it is well within the scope of the invention to provide for the situation where a number of different spacers may form part of the composition. Additionally, it should be understood that these structures do not necessarily represent a covalent bond between the peptides and the optional molecular spacer. As described above, the coupling interaction between the peptides and the optional spacer may be either covalent or non-covalent.

Compositions Comprising a Polymer-Binding Peptide

The peptide having affinity for a polymer may be applied to a body surface from various compositions, such as an aqueous solution or a personal care composition. For example, a polymer-binding peptide may be applied to the hair from an aqueous solution comprising the polymer-binding peptide. Alternatively, the polymer-binding peptide may be applied to the hair from a hair care composition (described below). In either case, the polymer-binding peptide is used in the composition at a concentration of about 0.01% to about 10%, preferably about 0.01% to about 5% by weight relative to the total weight of the composition. Suitable polymer-binding peptides are described above. Additionally, a mixture of different polymer-binding peptides may be used in the composition. The peptides in the mixture need to be chosen so that there is no interaction between the peptides that mitigates the beneficial effect. Suitable mixtures of polymer-binding peptides may be determined by one skilled in the art using routine experimentation. If a mixture of polymer-binding peptides is used in the composition, the total concentration of the polymer-binding peptides is about 0.01% to about 10% by weight relative to the total weight of the composition.

In another embodiment, a hair-binding peptide may be added to the composition comprising a polymer-binding peptide. The concentration of the hair-binding peptide in the composition is from about 0.01% to about 10%, preferably from about 0.01% to about 5%, relative to the total weight of the composition. Additionally, a mixture of different hair-binding peptides may be used. If a mixture of hair-binding peptides is used, the total concentration of the hair-binding peptides in the composition is from about 0.01% to about 10%, preferably from about 0.01% to about 5%, relative to the total weight of the composition.

In another embodiment, the polymer-binding peptide is used in the form of a peptide-based conjugate, wherein the polymer-binding peptide is coupled to a hair-binding peptide, as described above.

Hair care compositions are herein defined as compositions for the treatment of hair including, but not limited to, shampoos, conditioners, rinses, lotions, aerosols, gels, mousses, and hair dyes. The hair care composition may comprise a cosmetically acceptable medium for hair care compositions, examples of which are described for example by Philippe et al. in U.S. Pat. No. 6,280,747, and by Omura et al. in U.S. Pat. No. 6,139,851 and Cannell et al. in U.S. Pat. No. 6,013,250, all of which are incorporated herein by reference. For example, these hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hair care compositions may contain one or more conventional cosmetic or dermatological additives or adjuvants including, but not limited to, antioxidants, preserving agents, fillers, surfactants, UVA and/or UVB sunscreens, fragrances, thickeners, wetting agents and anionic, nonionic or amphoteric polymers, and dyes or pigments.

Similarly, the polymer-binding peptide may be applied to the skin from an aqueous solution comprising the polymer-binding peptide. Alternatively, the polymer-binding peptide may be applied to the skin from a skin care composition (described below). In either case, the skin-binding peptide is used in the composition at a concentration of about 0.01% to about 10%, preferably about 0.01% to about 5% by weight relative to the total weight of the composition. Suitable polymer-binding peptides are described above. Additionally, a mixture of different polymer-binding peptides may be used in the composition. The peptides in the mixture need to be chosen so that there is no interaction between the peptides that would mitigate the beneficial effect. Suitable mixtures of polymer-binding peptides may be determined by one skilled in the art using routine experimentation. If a mixture of polymer-binding peptides is used in the composition, the total concentration of the peptides is about 0.01% to about 10% by weight relative to the total weight of the composition.

In another embodiment, a skin-binding peptide may be added to the composition comprising a polymer-binding peptide. The concentration of the skin-binding peptide in the composition is from about 0.01% to about 10%, preferably from about 0.01% to about 5%, relative to the total weight of the composition. Additionally, a mixture of different skin-binding peptides may be used. If a mixture of skin-binding peptides is used, the total concentration of the skin-binding peptides in the composition is from about 0.01% to about 10%, preferably from about 0.01% to about 5%, relative to the total weight of the composition.

In another embodiment, the polymer-binding peptide is used in the form of a peptide-based conjugate, wherein the polymer-binding peptide is coupled to a skin-binding peptide, as described above.

Skin care compositions are herein defined as compositions for the treatment of skin including, but not limited to, skin care, skin cleansing, make-up, sunscreens, and anti-wrinkle products. The skin care composition may comprise a cosmetically acceptable medium for skin care compositions, examples of which are described for example by Philippe et al. supra. For example, the cosmetically acceptable medium may be an anhydrous composition containing a fatty substance in a proportion generally of from about 10 to about 90% by weight relative to the total weight of the composition, where the fatty phase containing at least one liquid, solid or semi-solid fatty substance. The fatty substance includes, but is not limited to, oils, waxes, gums, and so-called pasty fatty substances. Alternatively, the compositions may be in the form of a stable dispersion such as a water-in-oil or oil-in-water emulsion. Additionally, the compositions may contain one or more conventional cosmetic or dermatological additives or adjuvants including, but not limited to, antioxidants, preserving agents, fillers, surfactants, UVA and/or UVB sunscreens, fragrances, thickeners, wetting agents and anionic, nonionic or amphoteric polymers, and dyes or pigments.

Methods for Applying a Particulate Benefit Agent to a Body Surface

Polymer-binding peptides may be used to enhance the durability of common particulate benefit agents, for example, pigments, particulate conditioners, and inorganic sunscreens on body surfaces according to the method of the invention. For use in the invention, the particulate benefit agent is coated with a polymer, as described above. In general, the particulate benefit agent coated with a polymer is applied to the body surface either before, after, or concomitantly with a composition comprising a peptide having affinity for the polymer for a time sufficient for the coated benefit agent to bind to the body surface and the polymer-binding peptide to bind to the polymer coating on the particulate benefit agent. Various methods for applying a particulate benefit agent to hair or skin are described in more detail below.

In one embodiment, the particulate benefit agent coated with a polymer is applied to the hair. The coated benefit agent may be applied to the hair from any suitable solution, such as an aqueous solution or a conventional hair care composition, for example a coloring composition. These hair care compositions are well known in the art and suitable compositions are described above. The particulate benefit agent coated with a polymer is left on the hair for a time sufficient for the particulate benefit agent to bind to the hair, typically between about 5 seconds to about 60 minutes. Optionally, the hair may be rinsed to remove the particulate benefit agent that has not bound to the hair. Then, a composition comprising a peptide having affinity for the polymer coating is applied to the hair for a time sufficient for the polymer-binding peptide to bind to the polymer coating, preferably between about 5 seconds to about 60 minutes. The composition comprising the polymer-binding peptide may be rinsed from the hair or left on the hair.

In another embodiment, a composition comprising a peptide having affinity for the polymer coating is applied to the hair for a time sufficient for the polymer-binding peptide to bind to the hair, preferably between about 5 seconds to about 60 minutes. The unbound composition comprising the polymer-binding peptide may be rinsed from the hair or left on the hair. Then, the particulate benefit agent coated with a polymer is applied to the hair for a time sufficient for the particulate benefit agent to bind to the polymer-binding peptide, typically between about 5 seconds to about 60 minutes. Optionally, the hair may be rinsed to remove the particulate benefit agent that has not bound to the polymer-binding peptide.

In another embodiment, the particulate benefit agent coated with a polymer and the composition comprising a peptide having affinity for the polymer are applied to the hair concomitantly for a time sufficient for the particulate benefit agent to bind to hair and the polymer-binding peptide to bind to the polymer coating on the particulate benefit agent, typically between about 5 seconds to about 60 minutes. Optionally, the hair may be rinsed to remove the unbound particulate benefit agent and the composition comprising a polymer-binding peptide from the hair.

In another embodiment, the particulate benefit agent coated with a polymer is provided as part of the composition comprising a peptide having affinity for the polymer. In that embodiment, the composition comprising the particulate benefit agent and the polymer-binding peptide is applied to the hair for a time sufficient for the particulate benefit agent to bind to hair and the polymer-binding peptide to bind to the polymer coating on the particulate benefit agent, typically between about 5 seconds to about 60 minutes. The composition comprising the particulate benefit agent and the polymer-binding peptide may be rinsed from the hair or left on the hair.

In another embodiment, the particulate benefit agent coated with a polymer is applied to the skin. The coated benefit agent may be applied to the skin from any suitable solution, such as an aqueous solution or a conventional skin care composition, for example a skin colorant, skin conditioner, sunscreen, or the like, which is well known in the art. The particulate benefit agent coated with a polymer is left on the skin for a time sufficient for the particulate benefit agent to bind to the skin, typically between about 5 seconds to about 60 minutes. Optionally, the skin may be rinsed to remove the particulate benefit agent that has not bound to skin. Then, a composition comprising a peptide having affinity for the polymer coating on the particulate benefit agent is applied to the skin for a time sufficient for the polymer-binding peptide to bind to the polymer coating, preferably between about 5 seconds to about 60 minutes. The composition comprising the polymer-binding peptide may be rinsed from the skin or left on the skin.

In another embodiment, a composition comprising a peptide having affinity for the polymer coating, is applied to the skin for a time sufficient for the polymer-binding peptide to bind to the skin, preferably between about 5 seconds to about 60 minutes. The composition comprising the polymer-binding peptide may be rinsed from the skin or left on the skin. Then, the particulate benefit agent coated with a polymer is applied to the skin for a time sufficient for the particulate benefit agent to bind to the polymer-binding peptide, typically between about 5 seconds to about 60 minutes. Optionally, the skin may be rinsed to remove the particulate benefit agent that has not bound to the polymer-binding peptide.

In another embodiment, the particulate benefit agent coated with a polymer and the composition comprising a peptide having affinity for the polymer are applied to the skin concomitantly for a time sufficient for the particulate benefit agent to bind to skin and the polymer-binding peptide to bind to the polymer coating on the particulate benefit agent, typically between about 5 seconds to about 60 minutes. Optionally, the skin may be rinsed to remove the unbound particulate benefit agent and the composition comprising a polymer-binding peptide from the skin.

In another embodiment, the particulate benefit agent coated with a polymer is provided as part of the composition comprising a peptide having affinity for the polymer. In this embodiment, the composition comprising the particulate benefit agent and the polymer-binding peptide is applied to the skin for a time sufficient for the particulate benefit agent to bind to the skin and the polymer-binding peptide to bind to the polymer coating on the particulate benefit agent, typically between about 5 seconds to about 60 minutes. The composition comprising the particulate benefit agent and the polymer-binding peptide may be rinsed from the skin or left on the skin.

In any of the methods described above, the composition comprising a peptide having affinity for the polymer may optionally be reapplied to the body surface after the application of the composition comprising a particulate benefit agent coated with a polymer and the composition comprising a peptide having affinity for the polymer in order to further enhance the durability of the benefit agent.

Additionally, in any of the methods described above, a composition comprising a polymeric sealant may optionally be applied to the body surface after the application of the composition comprising a particulate benefit agent coated with a polymer and the composition comprising a peptide having affinity for the polymer in order to further enhance the durability of the benefit agent. The composition comprising the polymeric sealant may be an aqueous solution or a hair care or skin care composition comprising the polymeric sealant. Typically, the polymeric sealant is present in the composition at a concentration of about 0.25% to about 10% by weight based on the total weight of the composition. Polymeric sealants are well know in the art of personal care products and include, but are not limited to, poly(allylamine), acrylates, acrylate copolymers, methacrylates, methacrylate copolymers, polyurethanes, carbomers, methicones, amodimethicones, polypeptides, polyethylenene glycol, beeswax, siloxanes, and the like. The choice of polymeric sealant depends on the specific particulate benefit agent and the polymer-binding peptide used. The optimum polymeric sealant may be readily determined by one skilled in the art using routine experimentation.

Personal Care Compositions

The invention also provides personal care compositions comprising a particulate benefit agent coated with a polymer and a composition comprising a peptide having affinity for the polymer. The personal care composition may be any composition that is applied to a body surface to provide a cosmetic or prophylactic effect, such as the hair care and skin care compositions described above.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “sec” means second(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmole” means micromole(s), “g” means gram(s), “μg” means microgram(s), “mg” means milligram(s), “g” means the gravitation constant, “rpm” means revolutions per minute, “pfu” means plague forming unit, “BSA” means bovine serum albumin, “ELISA” means enzyme linked immunosorbent assay, “IPTG” means isopropyl β-D-thiogalactopyranoside, “A” means absorbance, “A₄₅₀” means the absorbance measured at a wavelength of 450 nm, “TBS” means Tris-buffered saline, “TBST-X” means Tris-buffered saline containing Tween® 20 where “X” is the weight percent of Tween®20, “Xgal” means 5-bromo-4-chloro-3-indolyl-beta-D-galactopyranoside, “SEM” means standard error of the mean, “vol %” means volume percent, “atm” means atmosphere(s), “kPa” means kilopascal(s), “SLPM” means standard liter per minute, “psi” means pounds per square inch, “RCF” means relative centrifugal field.

General Methods:

Standard recombinant DNA and molecular cloning techniques used in the Examples are well known in the art and are described by Sambrook, J., Fritsch, E. F. and Maniatis, T., Molecular Cloning: A Laboratory Manual, Cold Spring Harbor Laboratory Press, Cold Spring Harbor, N.Y., 1989, by T. J. Silhavy, M. L. Bennan, and L. W. Enquist, Experiments with Gene Fusions, Cold Spring Harbor Laboratory, Cold Spring Harbor, N.Y., 1984, and by Ausubel, F. M. et al., Current Protocols in Molecular Biology, Greene Publishing Assoc. and Wiley-lnterscience, N.Y., 1987. Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989.

All reagents, restriction enzymes and materials used for the growth and maintenance of bacterial cells were obtained from Aldrich Chemicals (Milwaukee, Wis.), BD Diagnostic Systems (Sparks, Md.), Life Technologies (Rockville, Md.), or Sigma Chemical Company (St. Louis, Mo.), unless otherwise specified.

Phage Display Peptide Libraries:

Six phage display peptide libraries were used in the following Examples. Three peptide libraries, Ph.D.™-12 Phage Display Peptide Library Kit (a 12-mer linear peptide library), Ph.D.™-7 Phage Display Library Kit (a 7-mer linear peptide library), and Ph.D.™-C7C Phage Display Library Kit (a 7-mer constrained peptide library), were purchased from New England BioLabs (Beverly, Mass.). These kits are based on a combinatorial library of random peptide 7 or 12-mers fused to a minor coat protein (pill) of M13 phage. The randomized peptide sequences in all three libraries are expressed at the N-terminus of the minor coat protein pill, resulting in a valency of 5 copies of the displayed peptide per virion. In both the Ph.D.-7 and the Ph.D.-12 libraries, the first residue of the peptide-pill fusion is the first randomized position, while the first randomized position in the Ph.D.-C7C library is preceded by Ala-Cys. All of the libraries contain a short linker sequence of four amino acids between the displayed peptide and pill. The randomized segment of the Ph.D.-C7C library is flanked by a pair of cysteine residues, which are oxidized during phage assembly to a disulfide linkage, resulting in the displayed peptides being presented to the target as loops. All three libraries consist of approximately 3×10⁹ sequences. A volume of 10 μL contains about 55 copies of each peptide sequence.

Three other phage display peptide libraries, one containing 15-mer random linear peptide sequences, another containing 20-mer random linear peptide sequences, and a third containing 14-mer disulfide constrained random peptide sequences with a cystine residue at positions 3 and 11, were prepared using the method described by Kay et al. (Combinatorial Chemistry & High Throughput Screening, Vol. 8, 545-551 (2005)). This method is a modification of the method reported by Sidhu et al. (Methods in Enzymology 328:333-363 (2000)) in which E. coli strain CJ236 (dut⁻ ung⁻) is used to generate uridine-containing single-stranded phagemid DNA (U-ssDNA). This DNA was used as a template for second-strand synthesis using an oligonucleotide, not only as a primer of the second strand, but also to insert encoding random amino acids. Upon completion of second strand synthesis, the double stranded (dsDNA) was transformed into a wild-type strain. Any U-ssDNA was degraded by the host cell, thus leaving only the recombinant strand to generate phage particles. This method can be utilized to generate peptide fusions or mutations to the M13 coat proteins. The method of Kay et al. uses an amber stop codon at beginning of gene III. Oligonucleotides containing randomized stretches of DNA sequence were annealed to the single-stranded phage genome, such that the randomized region aligns with the stop codon. The single stranded DNA (ssDNA) was enzymatically converted to covalently-closed, circular dsDNA and subsequently electroporated into a non-suppressor strain of E. coli. The newly synthesized DNA strand (minus strand) served as the template for generation of the plus strand in the host cell, which was utilized for transcription/translation of viral genes and was packaged into the virus particle.

### Example 1

### Selection of Polymethylmethacrylate (PMMA)-Binding Peptides Using Biopanning

The purpose of this Example was to identify phage peptides that bind to polymethylmethacrylate (PMMA) using a modified phage display biopanning method.

The Ph.D.™-12 Phage Display Peptide Library Kit and Ph.D.™-7 Phage Display Library Kit were used in this Example. Each initial round of experiments was carried out using the original library provided by the manufacturer in order to avoid introducing any bias into the results.

Biopanning Against a PMMA Surface:

The PMMA materials used were ⅛ inch (32 mm) thick, ½ inch (12.7 mm) diameter disks of Lucite® methyl methacrylate polymer sheet (obtained from E.I. du Pont de Nemours and Co., Wilmington, Del.) and a dot blot apparatus (obtained from Schleicher & Schuell, Keene, N.H.). The following protocol was used for biopanning against the PMMA disk. The PMMA disk was placed in a tube filled with 5 mL of 90% isopropanol for 30 min at room temperature and then washed 5 times for 10 min each with deionized water. Then, 5 mL of blocking buffer consisting of 1 mg/mL BSA in TBST containing 0.5% Tween® 20 (TBST-0.5%) was added to the tube and incubated for 1 h at 4° C.

The disk was washed 5 times with TBST-0.5% and then 2 mL of TBST-0.5% containing 1 mg/mL BSA was added to each well. Then, 10 μL of the original phage library (2×10¹¹ pfu), either the 12-mer or 7-mer library, was added to the disk and incubated for 15 min at room temperature. The disk was washed 10 times with TBST-0.5%. The disk was then transferred to a clean tube, 2 mL of a non-specific elution buffer consisting of 1 mg/mL BSA in 0.2 M glycine-HCl, pH 2.2, was added to the tube and incubated for 10 min. The disk was washed three more times with the elution buffer and then washed three times with TBST-0.5%. The disk, which had acid resistant phage peptides still attached, was used to directly infect the host cells E. coli ER 2738 (New England BioLabs, Beverly, Mass.), for phage amplifications. The disk was incubated with an overnight E. coli ER2738 culture diluted 1:100 in LB (Luria-Bertani) medium, at 37° C. for 4.5 h. After this time, the cell culture was centrifuged for 30 s and the upper 80% of the supernatant was transferred to a fresh tube, ⅙ volume of PEG/NaCl (20% polyethylene glycol-800, obtained from Sigma Chemical Co. St. Louis, Mo., 2.5 M sodium chloride) was added, and the phage was allowed to precipitate overnight at 4° C. The precipitate was collected by centrifugation at 10,000×g at 4° C. and the resulting pellet was resuspended in 1 mL of TBS. This amplified first round phage stock was then titered according to the method described below. For the next round of biopanning, more than 2×10¹¹ pfu of phage stock from the first round was used. The biopanning process was repeated for 3 to 4 rounds depending on the experiments.

After the acid wash steps in the final round of biopanning, the PMMA disk was used to directly infect 500 μL of mid-log phase bacterial host cells, E. coli ER2738, which were then grown in LB medium for 20 min and then mixed with 3 mL of agarose top (LB medium with 5 mM MgCl₂, and 0.7% agarose) at 45° C. This mixture was spread onto a LB medium/lPTG/ S-Gal™ plate (LB medium with 15 g/L agar, 0.05 g/L IPTG, and 0.04 g/L S-Gal™) and incubated overnight at 37° C. The black plaques were counted to calculate the phage titer. Single black plaques were randomly picked for DNA isolation and sequencing analysis. The amino acid sequences of these high affinity, PMMA-binding phage peptides are given in Table 1.

TABLE 1

Amino Acid Seauences of High Affinity PMMA-Binding
Phage Peptides from the 7- and 12-Mer Libraries

| | | |
|---|---|---|
| Clone ID | Amino Acid Sequence | SEQ ID NO: |

| | | |
|---|---|---|
| A09 | IPWWNIRAPLNA | 1 |
| D09 | TAVMNWNNQLS | 2 |
| A03 | VPWWAPSKLSMQ | 3 |
| A06 | MVMAPHTPRARS | 4 |
| B04 | TYPNWAHLLSHY | 5 |
| B09 | TPWWRIT | 6 |
| B01 | DLTLPFH | 7 |
| PB411 | GTSIPAM | 8 |
| P307 | HHKHWA | 9 |
| P410 | HHHKHFM | 10 |
| P202 | HHHRHQG | 11 |
| PNM407 | HHWHAPR | 12 |

### Example 2

### Characterization of PMMA-Binding Phage Peptide Clones by ELISA

Enzyme-linked immunosorbent assay (ELISA) was used to evaluate the PMMA-binding affinity of the selected phage-peptide clones identified in Example 1 along with a skin-1 phage clone TPFHSPENAPGS (given as SEQ ID NO:53), which served as a control.

An empty 96-well apparatus, a Minifold I Dot-Blot System from Schleicher & Schuell, Inc. (Keene, N.H.) was used as the PMMA surface. For each clone tested, the well was incubated for 1 h at room temperature with 200 μL of blocking buffer, consisting of 2% non-fat dry milk in TBS. The blocking buffer was removed by inverting the systems and blotting them dry with paper towels. The wells were rinsed 6 times with wash buffer consisting of TBST-0.5%. The wells were filled with 200 μL of TBST-0.5% containing 1 mg/mL BSA and then 10 μL (over 10¹² copies) of purified phage stock was added to each well. The samples were incubated at 37° C. for 15 min with slow shaking. The non-binding phage was removed by washing the wells 10 to 20 times with TBST-0.5%. Then, 100 μL of horseradish peroxidase/anti-M13 antibody conjugate (Amersham USA, Piscataway, N.J.), diluted 1:500 in the blocking buffer, was added to each well and incubated for 1 h at room temperature. The conjugate solution was removed and the wells were washed 6 times with TBST-0.05%. TMB substrate (200 μL), obtained from Pierce Biotechnology (Rockford, Ill.) was added to each well and the color was allowed to develop for between 5 to 30 min, typically for 10 min, at room temperature. Then, stop solution (200 μL of 2 M H₂SO₄) was added to each well and the solution was transferred to a 96-well plate and the A₄₅₀ was measured using a microplate spectrophotometer (Molecular Devices, Sunnyvale, Calif.). The resulting absorbance values, reported as the mean of at least three replicates, and the standard error of the mean (SEM) are given in Table 2.

TABLE 2
Results of ELISA Assay

| | SEQ ID | PMMA | |
|---|---|---|---|
| Clone ID | NO: | A₄₅₀ | SEM |
| Skin-1 | 53 | 0.127 | 0.057 |
| (Control) | | | |
| A09 | 1 | 2.227 | 0.020 |
| D09 | 2 | 2.037 | 0.057 |
| A03 | 3 | 0.762 | 0.081 |
| A06 | 4 | 2.09 | 0.115 |
| B04 | 5 | 2.095 | 0.065 |
| B09 | 6 | 2.261 | 0.016 |
| B01 | 7 | 2.112 | 0.060 |

The results demonstrate that all of the PMMA-binding phage peptides tested had a significantly higher binding affinity for PMMA than the control skin-1 peptide.

### Example 3

### Determination of the PMMA-Binding Affinity of PMMA-Binding Peptides

The purpose of this Example was to determine the affinity of the PMMA-binding peptides for PMMA surfaces, measured as MB50 values, using an ELISA assay.

The PMMA-binding peptide, A09, was synthesized by Synpep Inc. (Dublin, Calif.). The peptide was biotinylated by adding a biotinylated lysine residue at the C-terminus of the amino acid binding sequence for detection purposes and an amidated cysteine was added to the C-terminus of the sequence. The amino acid sequence of the peptide tested is given as SEQ ID NO:13.

MB₅₀ Measurement of PMMA-Binding Peptide A09:

The MB₅₀ measurements of biotinylated peptide A09 (SEQ ID NO:13) binding to PMMA were done using the 96-well apparatus described in Example 2. The 96-wells were blocked with blocking buffer (SuperBlock™ from Pierce Chemical Co., Rockford, Ill.) at room temperature for 1 h, followed by six washes with TBST-0.5%, 2 min each, at room temperature. Various concentrations of biotinylated, binding peptide were added to each well, incubated for 15 min at 37° C., and washed six times with TBST-0.5%, 2 min each, at room temperature. Then, streptavidin-horseradish peroxidase (HRP) conjugate (Pierce Chemical Co., Rockford, Ill.) was added to each well (1.0 μg per well), and incubated for 1 h at room temperature. After the incubation, the wells were washed six times with TBST-0.5%, 2 min each at room temperature. Finally, the color development and the absorbance measurements were performed as described in Example 2.

The results were plotted as A₄₅₀ versus the concentration of peptide using GraphPad Prism 4.0 (GraphPad Software, Inc., San Diego, Calif.). The MB₅₀ values were calculated from Scatchard plots and are shown Table 3.

MB₅₀ Measurement of Tetramer PMMA-Binding Peptide A09:

For the MB₅₀ measurement of the peptide A09 tetramer, the PMMA surface and all the binding conditions were the same as described above. The tetrameric-A09 peptide complex was prepared by mixing streptavidin-HRP and biotinylated peptide A09 (SEQ ID NO:13) in a 1:4 molar ratio. After all the blocking and washing steps, various concentrations of Streptavidin/(A09)₄ complex were added to each well, incubated for 15 min at 37° C., and washed six times with TBST-0.5%, 2 min each, at room temperature. Then, color development and the absorbance measurements were performed as described in Example 2. The results were plotted as A₄₅₀ versus the concentration of peptide complex using GraphPad Prism 4.0 (GraphPad Software, Inc., San Diego, Calif.). The MB₅₀ values were calculated from Scatchard plots and are shown in Table 3.

TABLE 3
MB₅₀ Values for Selected PMMA-Binding Peptides

| Binding | Peptide Sequence | | |
|---|---|---|---|
| Peptide | Tested | Substrate | MB_{50,} M |
| A09 | SEQ ID NO: 13 | PMMA | 5.9 × 10⁻⁸ |
| Streptavidin/ | (SEQ ID NO: 13)₄ | PMMA | 3.9 × 10⁻⁹ |
| (A09)₄ | | | |

The results demonstrate that the binding affinity of the PMMA-binding peptide, A09, and the straptavidin/A09 tetramer complex for PMMA was high. The use of multiple copies of the binding peptide in the A09 tetramer complex increased the binding affinity by more than 10-fold.

### Example 4

### Hair Coloring Using a Pigment Dispersed with a Methyacrylic Acid-Containing Polymeric Dispersant in Coniunction with a PMMA-Binding Peptide

The purpose of this Example was to demonstrate hair coloring using a carbon black pigment, dispersed with a methacrylic acid-containing polymeric dispersant, in conjunction with a PMMA-binding peptide.

Preparation of Carbon Black Dispersion:

A carbon black dispersion was prepared by first mixing well the following ingredients: (i) 210.4 parts by weight (pbw) deionized water, (ii) 80.3 pbw of a 41.5 wt % (solids) anionic polymeric dispersant, and (iii) 9.24 pbw of dimethylethanolamine. The anionic polymer dispersant was a graft co-polymer 66.3/-g-4.2/29.5 POEA(phenoxyethyl acrylate)/-g-ETEGMA(ethoxytriethyleneglycolmethacrylate)/MAA (methacrylic acid) prepared according to “Preparation of Dispersant 1” in U.S. Patent Application Publication No. 20030128246 (paragraphs 0122 through 0125), which is incorporated herein by reference, with the ratios of monomers adjusted to obtain the 66.3/4.2/29.5 percent by weight ratios instead of the 61.6/5.8/32.6 percent by weight ratios indicated in the publication. To this was gradually added 100 pbw black pigment (Nipex 180IQ, Degussa). After the pigment was incorporated, 100 pbw deionized water was mixed in to form the millbase, which was circulated through a media mill for grinding. Deionized water (55.4 pbw) was then added for dilution to final strength. This black dispersion (276 g) was mixed with 200 g of glycerol, 120 g of ethylene glycol, 1.6 g of Proxel (Arch Chemicals, Inc., Cheshire, Conn.) 143.6 g of deionized water and 2.5 g of Surfynol® 485 (Air Products, Allentown, Pa.) to form a black colorant base. The pH of the colorant formulations was adjusted to 7.0, as needed, by addition of 10% phosphoric acid or 10% sodium hydroxide solution. Aqueous carbon black stock solutions were prepared by dilution of this colorant base with water to achieve carbon loadings of 1% to 2% by weight for use in hair durability studies.

The A09 PMMA-binding peptide having an amidated cysteine added to the C-terminal end, given as SEQ ID NO:14, was obtained from SynPep (Dublin, Calif.). This peptide (100 mg) was added to 10 g of a 1% carbon black dispersion and the solution was stirred for several hours at room temperature.

Hair Coloring:

A hair swatch of natural white hair (2.60 g), obtained from International Hair Importers and Products (Bellerose, N.Y.), was placed in a 3 mm×100 mm test tube and 7-8 mL of the carbon black dispersion containing the PMMA binding peptide was added. The mixture was stirred using a magnetic stirrer for 30 min to ensure good contact of the pigment dispersion with the hair. The hair swatch was removed from the test tube and allowed to air dry for 30 min.

The durability of the hair color was tested by rinsing the hair with deionized water. The hair swatch was massaged by hand during the water rinse. After the water rinse, the hair swatch retained most of the black color.

### Example 5 (Comparative)

### Hair Coloring Using a Pigment Dispersed with a Methacrylic Acid-Containing Polymeric Dispersant in the Absence of a PMMA Binding Peptide

The purpose of this Example was to assess the durability of the hair coloring obtained using the carbon black pigment in the absence of the PMMA-binding peptide and to compare it to the durability obtained in Example 4.

The carbon black dispersion was prepared as described in Example 4, but the PMMA binding peptide was not added. The hair coloring and water rinsing were done as described in Example 4 using 10 g of the carbon black dispersion and a 2.30 g hair swatch.

After the water rinse, only a trace of the black color remained. When compared to the result obtained in Example 4, this result demonstrates the improved durability obtained when the carbon black pigment is used in conjunction with a PMMA binding peptide.

### Example 6 (Comparative)

### Hair Coloring Using a Pigment Dispersed with a Methacrylic Acid-Containing Polymeric Dispersant in Conjunction with a Siloxane Sealant

The purpose of this Example was to assess the durability of the hair coloring obtained using the carbon black pigment in conjunction with a conventional siloxane sealant and to compare it to the durability obtained in Example 4.

The carbon black dispersion was prepared as described in Example 4, but the PMMA binding peptide was not added. One gram of decamethylcyclopentasiloxane (Aldrich. Milwaukee, Wis.; Product No. 44,427-8; CAS No. 541-02-6) was added to 9 g of the carbon black dispersion. The hair coloring and water rinsing were done as described in Example 4 using this carbon black dispersion and a 2.30 g hair swatch.

After the water rinse, all of the black color was washed out. When compared to the result obtained in Example 4, this result demonstrates the improved durability obtained with the carbon black pigment used in conjunction with a PMMA binding peptide compared to the pigment used with a conventional siloxane sealant.

### Example 7

### Hair Coloring Usinq a Pigment Dispersed with a Methacrylic Acid-Containing Polymeric Dispersant in Conjunction with a PMMA-Binding Peptide and a Poly(allylamine) Sealant

The purpose of this Example was to demonstrate the enhanced durability of hair color obtained with the use of poly(allylamine) as a polymeric sealant in conjunction with the carbon black pigment and a PMMA-binding peptide.

The carbon dispersion was prepared as described in Example 4 and was used with the PMMA binding peptide. The hair coloring was done as described in Example 4 using a 2.66 g hair swatch. After the hair swatch was colored and dried for 30 min, the hair sample was dipped in a 4 wt % solution of poly(allylamine) (prepared by dilution with water of a 20 wt % aqueous solution of poly(allylamine), obtained from Aldrich; Product No. 479177; CAS No. 30551-89-4). The hair swatch was immediately rinsed as described in Example 4 and then treated with shampoo (Pantene Pro-V Sheer Volume, Proctor & Gamble, Cincinnati, Ohio). The shampoo treatment involved the addition of a quarter-sized drop of the shampoo to the hair, distributing the shampoo evenly over the hair, and aggressively massaging the hair for 30 sec. Then, the hair swatch was rinsed with deionized water to remove the shampoo.

After the water rinse and the shampoo treatment, the hair swatch retained approximately 50% of the black color. The result that the color survived a vigorous shampoo treatment demonstrates that the use of poly(allylamine) as a sealant in conjunction with the carbon black pigment and the PMMA binding peptide provides enhanced durability compared to that obtained with the carbon black pigment and the PMMA-binding peptide alone.

### Examples 8-12

### Hair Coloring Using a Pigment Dispersed with a Methacrylic Acid-Containing Polymeric Dispersant in Conjunction with a PMMA-Binding Peptide

The purpose of these Examples was to demonstrate the enhanced durability of hair color obtained using a carbon black pigment, dispersed with a methacrylic acid-containing polymeric dispersant, in conjunction with a PMMA-binding peptide and to compare it to the durability obtained with the carbon black pigment alone. Additionally, the additional enhancement provided by a poly(allylamine) sealant application was demonstrated. The color retention was quantified using a spectrophotometic measurement technique.

The carbon black dispersion was prepared as described in Example 4. The coloring of the hair was done as described in Example 4 with and without the use of the PMMA-binding peptide (SEQ ID NO:14). The effect of a poly(allylamine) sealant was tested as described in Example 7.

The color intensity after a water rinse (done as described in Example 4) and after shampoo treatment (done as described in Example 7) was measured using a X-Rite® SP78™ Sphere Spectrophotometer (X-Rite, Inc., Grandville, Mich.), by placing the colored hair sample into the photosensor and calculating L*, a* and b* parameters representing the photometer response. An initial baseline L* value was measured for the uncolored hair and all measurements were the average of three individual determinations. Delta E values were calculated using equation 1 below:
Delta E=((L*₁−L*₂)²+(a₁−a₂)²+(b₁−b₂)²)^{1/2} (1)

where L*=the lightness variable and a* and b* are the chromaticity coordinates of CIELAB colorspace as defined by the International Commission of Illumination (CIE) (Minolta, Precise Color Communication—Color Control From Feeling to Instrumentation, Minolta Camera Co., 1996). Larger Delta E value are indicative of better color retention. The results are summarized in Table 4.

TABLE 4
Results of Hair Color Durability Testing

| | Coloring | | |
|---|---|---|---|
| Example | Conditions | Treatment | Delta E |
| 8 | Carbon Black + PMMA- | Water Rinse | 30.7 |
| | Binding | | |
| | Peptide | | |
| 9, Comparative | Carbon Black | Water Rinse | 2.58 |
| 10 | Carbon Black + PMMA- | Shampoo | 7.28 |
| | Binding | | |
| | Peptide | | |
| 11 | Carbon Black + PMMA- | Shampoo | 23.5 |
| | Binding | | |
| | Peptide + Poly | | |
| | (allylamine) | | |
| | Sealant | | |
| 12, Comparative | Carbon Black | Shampoo | 0.84 |

The results demonstrate that the use of the PMMA-binding peptide in conjunction with the methacrylate-coated carbon black pigment resulted in a significant enhancement of the retention of the hair color after both a water rinse (Example 8) and the shampoo treatment (Example 10) compared to the use of carbon black alone (Comparative Examples 9 and 12). Additionally, the use of the poly(allylamine) sealant provided further enhancement in the color retention following the shampoo treatment (Example 11).

### Example 13

### Selection of Polypropylene-Binding Peptides Using Biopanning

The purpose of this Example was to identify phage peptides that bind to polypropylene (PP) using a modified phage display biopanning method.

The polypropylene-binding peptides were identified using the biopanning method described in Example 1. The polypropylene substrate used in the biopanning method was a polypropylene mesh material, specifically, Hernia Repair/Reconstructive Surgery Prosthetics Patch (obtained from Davol Inc., Cranston, R.I., a subsidiary of C.R. Bard Inc.). The polypropylene mesh was cut into 1-cm squares and pretreated with 90% isopropanol for 30 min at room temperature, followed by washing 5 times for 10 min each with deionized water before the panning process. A total of four rounds of biopanning were performed and the amino acid sequences of the high affinity, polypropylene-binding phage peptides are given in Table 5.

TABLE 5

Amino Acid Sequences of High Affinity PP-Binding
Phage Peptides from the 7- and 12-Mer Libraries

| | |
|---|---|
| Amino Acid Sequence | +HZ,17 SEQ ID NO: |

| | |
|---|---|
| TSDIKSRSPHHR | 15 |
| HTQNMRMYEPWF | 16 |
| LPPGSLA | 17 |
| MPAVMSSAQVPR | 18 |
| NQSFLPLDFPFR | 19 |
| SILSTMSPHGAT | 20 |
| SMKYSHSTAPAL | 21 |

### Example 14

### Selection of Polytetrafluoroethylene-Binding Peptides Using Biopanning

The purpose of this Example was to identify phage peptides that bind to polytetrafluoroethylene (PTFE) using a modified phage display biopanning method.

The polytetrafluoroethylene-binding peptides were identified using the biopanning method described in Example 1. The polytetrafluoroethylene substrate used in the biopanning method was a ePTFE film material (obtained from Davol Inc., Cranston, R.I., a subsidiary of C.R. Bard Inc.). The polytetrafluoroethylene film was cut into 1-cm squares and pretreated with 90% isopropanol for 30 min at room temperature, followed by washing 5 times for 10 min each with deionized water before the panning process. A total of four rounds of biopanning were performed and the amino acid sequences of the high affinity, PTFE-binding phage peptides are given in Table 6.

TABLE 6

Amino Acid Sequences of High Affinity PTFE-Binding
Phage Peptides from the 7- and 12-Mer Libraries

| Amino Acid Sequence | SEQ ID NO: |
|---|---|
| ESSYSWSPARLS | 22 |
| GPLKLLHAWWQP | 23 |
| NALTRPV | 24 |
| SAPSSKN | 25 |
| SVSVGMKPSPRP | 26 |
| SYYSLPPIFHIP | 27 |
| TFTPYSITHALL | 28 |
| TMGFTAPRFPHY | 29 |
| TNPFPPPPSSPA | 30 |

### Example 15

### Selection of Nylon 6.6-Binding Peptides Using Biopanning

The purpose of this Example was to identify phage peptides that bind to nylon 6,6 using a modified phage display biopanning method.

The nylon 6,6-binding peptides were identified using the biopanning method described in Example 1. Nylon 6,6, beads used as the substrate in the biopanning method were additive free and were prepared using standard nylon polymerization processes that are well known in the art (See Kohan, M.I., Nylon Plastics Handbook, Hansen/Gardner Publications, Inc. [1995] pages 17-20 & 34-45). The nylon beads were pretreated with 90% isopropanol for 30 min at room temperature, followed by washing 5 times for 10 min each with deionized water before the biopanning process. A total of four rounds of biopanning were performed and the amino acid sequences of the high affinity, nylon 6,6-binding phage peptides are given in Table 7.

TABLE 7

Amino Acid Sequences of High Affinity
Nylon-Binding Phage Peptides from the
7-Mer Library

| | |
|---|---|
| Amino Acid Sequence | SEQ ID NO: |

| | |
|---|---|
| KTPPTRP | 31 |
| VINPNLD | 32 |
| KVWIVST | 33 |
| AEPVAML | 34 |
| AELVAML | 35 |
| HSLRLDW | 36 |

### Example 16

### Selection of Polyethylene-Bindinq Peptides Using Biopanning

The purpose of this Example was to identify phage peptides that bind to polyethylene (PE) using a modified phage display biopanning method.

The polyethylene-binding peptides were identified using the biopanning method described in Example 1. Ultra high molecular weight polyethylene tape was used as the substrate in the biopanning method. A similar tape may be obtained from Davol, Inc, (Cranston, R.I.). The PE tape was cut into 1-cm squares and the squares were pretreated with 90% isopropanol for 30 min at room temperature, followed by washing 5 times for 10 min each with deionized water before biopanning. A total of four rounds of biopanning were performed and the amino acid sequences of the high affinity, PE-binding phage peptides are given in Table 8.

TABLE 8

Amino Acid Sequences of High Affinity PE-Binding
Phage Peptides from the 12-Mer Library

| | |
|---|---|
| Amino Acid Sequence | SEQ ID NO: |

| | |
|---|---|
| HNKSSPLTAALP | 37 |
| LPPWKHKTSGVA | 38 |
| LPWWLRDSYLLP | 39 |
| VPWWKHPPLPVP | 40 |
| HHKQWHNHPHHA | 41 |
| HIFSSWHQMWHR | 42 |
| WPAWKTHPILRM | 43 |

### Example 17

### Selection of Polystyrene-Binding Peptides Using Biopanning

The purpose of this Example was to identify phage peptides that bind to polystyrene (PS) using a modified phage display biopanning method.

The polystyrene-binding peptides were identified using the biopanning method described in Example 1. The polystyrene (PS)-binding peptides were discovered during biopanning experiments against soluble Type I collagen-coated 96-well polystyrene plates (Corning Inc., Acton, MA). The 96-wells were coated with 100 ng/ml of soluble type I collagen (Sigma-Aldrich, St Louis, Mo.). A total of four rounds of biopanning were performed. Three highly enriched phage peptides were identified that were later confirmed to bind to the PS well, not the coated type I collagen. The amino acid sequences of the high affinity, PS-binding phage peptides are given in Table 9.

TABLE 9

Amino Acid Seauences of High Affinity PS-Binding
Phage Peptides from the 12-Mer Library

| | |
|---|---|
| Amino Acid Sequence | SEQ ID NO: |

| | |
|---|---|
| TSTASPTMQSKIR | 44 |
| KRNHWQRMHLSA | 45 |
| SHATPPQGLGPQ | 46 |

### Example 18

### Biological Production of a Triblock Peptide-Based Conjugate

The purpose of this Example was to prepare a triblock peptide-based conjugate using recombinant DNA and molecular cloning techniques. The triblock peptide-based conjugate was comprised of multiple hair-binding peptide, peptide spacer, and PMMA-binding peptide blocks. The peptides were expressed in E. coli as inclusion bodies. Additional amino acid sequences (i.e., peptide tags) were fused to the triblock peptide-based conjugate sequence in order to promote inclusion body formation. Acid-labile Asp-Pro (DP) sequences were placed between the peptide tag and the triblock peptide-based conjugate sequence to facilitate isolation of the triblock peptide from the peptide tag.

Construction of Production Strains

The sequences of the triblock peptide-based conjugate is given in Table 10. DNA sequences were designed to encode this peptide sequence using favorable codons for E. coli and to avoid sequence repeats and mRNA secondary structure. The gene DNA sequence was designed by DNA 2.0, Inc. (Menlo Park, Calif.) using proprietary software which is described by Gustafsson et al. (Trends in Biotechnol. 22(7):346-355 (2004)). The sequence encoding the amino acid sequence was followed by two termination codons and a recognition site for endonuclease Ascl. The GS amino acid sequence at the N-terminus was encoded by a recognition site for endonuclease BamHl (GGA/TCC). The DNA sequence is given by SEQ ID NO:71.

TABLE 10

Peptide Sequence and DNA Encoding Sequence of
Triblock Peptide-Based Conjugate

| Peptide | | | Peptide | DNA |
|---|---|---|---|---|
| Conjugate | Peptide Sequence | DNA Sequence* | SEQ ID NO: | SEQ ID NO: |
| HC77643 | PG (Spacer)- | GGATCCGACCCTGGT | 70 | 71 |
| | IPWWNIRAPLNA | ATCCCGTGGTGGAACA | | |
| | (PMMA-binding | TTCGCGCACCTCTGAA | | |
| | peptide)- GAG | TGCTGGTGCTGGTATT | | |
| | (spacer)- | CCGTGGTGGAACATC | | |
| | IPWWNIRAPLNA | CGTGCTCCTCTGAACG | | |
| | (PMMA-binding | CGGGTGGCTCCGGTC | | |
| | peptide)- | ACACGAGCCAACTGA | | |
| | GGSGPGSGG | GCACCGGTGGTGGCA | | |
| | (spacer)- | ACACTTCCCAGCTGTC | | |
| | NTSQLST (hair- | CACCGGCGGTCCGAA | | |
| | binding peptide)- | AAAGTAATAAGGCGCG | | |
| | GGG (spacer)- | CC | | |
| | NTSQLST (hair- | | | |
| | binding peptide)- | | | |
| | GGPKK (spacer) | | | |

*The coding sequence for the peptide conjugate is underlined.

The genes was assembled from synthetic oligonucleotides and cloned into a standard plasmid cloning vector by DNA 2.0, Inc. The sequences was verified by DNA sequencing by DNA 2.0, Inc.

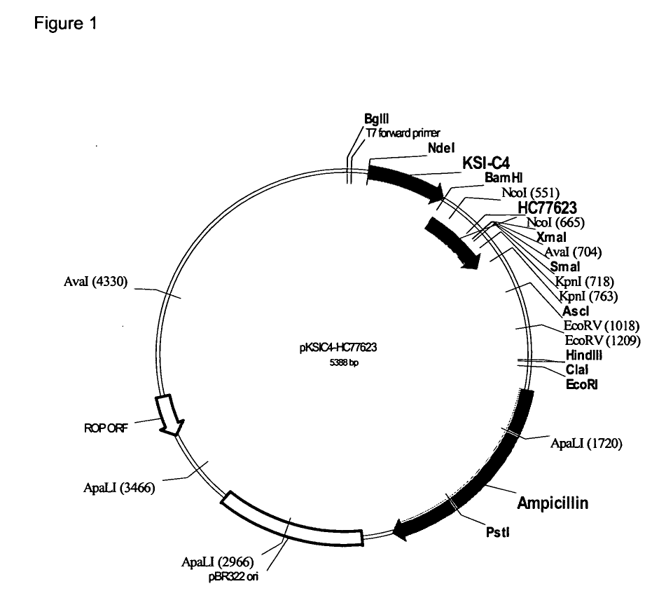

The synthetic gene was excised from the cloning vector with the endonuclease restriction enzymes BamHl and Ascl and ligated into an expression vector using standard recombinant DNA methods. The vector pKSIC4-HC77623 was derived from the commercially available vector pDEST17 (Invitrogen, Carlsbad, calif.). It includes sequences derived from the commercially available vector pET31 b (Novagen, Madison, Wis.) that encode a fragment of the enzyme ketosteroid isomerase (KSI). The KSI fragment was included as a fusion partner to promote partition of the peptides into insoluble inclusion bodies in E. coli. The KSI-encoding sequence from pET31b was modified using standard mutagenesis procedures (QuickChange II, Stratagene, La Jolla, calif.) to include three additional Cys codons, in addition to the one Cys codon found in the wild type KSI sequence. The plasmid pKSIC4-HC77623, given by SEQ ID NO:72 and shown in FIG. 1, was constructed using standard recombinant DNA methods, which are well known to those skilled in the art.

The DNA sequence encoding the triblock peptide-based body conjugate (Table 1) was inserted into pKSIC4-HC77623 by substituting for sequences in the vector between the BamHl and Ascl sites. Plasmid DNA containing the peptide encoding sequence and vector DNA was digested with endonuclease restriction enzymes BamHl and Ascl, then the peptide-encoding sequence and vector DNA were mixed and ligated by phage T4 DNA ligase using standard DNA cloning procedures, which are well known to those skilled in the art. The correct construct, in which the sequence encoding the triblock peptide-based conjugate was inserted into pKSIC4-HC77623, was identified by restriction analysis and verified by DNA sequencing, using standard methods.

In this construct, the sequence encoding the peptide conjugate was substituted for those encoding HC77623. The sequence was operably linked to the bacteriophage T7 gene 10 promoter and expressed as a fusion protein, fused with the variant KSI partner.

To test the expression of the peptide-based conjugate, the expression plasmid was transformed into the BL21-AI E. coli strain (Invitrogen, catalog no. C6070-03). To produce the recombinant fusion peptide, 50 mL of LB-ampicillin broth (10 g/L bacto-tryptone, 5 g/L bacto-yeast extract, 10 g/L NaCI, 100 mg/L ampicillin, pH 7.0) was inoculated with the transformed bacteria and the culture was shaken at 37° C. until the OD₆₀₀ reached 0.6. The expression was induced by adding 0.5 mL of 20 wt % L-arabinose to the culture and shaking was continued for another 4 h. Analysis of the cell protein by polyacrylamide gel electrophoresis demonstrated the production of the fusion peptides.

Fermentation:

The recombinant E. coli strain, described above, was grown in a 6-L fermentation, which was run in batch mode initially, and then in fed-batch mode. The composition of the fermentation medium is given in Table 11. The pH of the fermentation medium was 6.7. The fermentation medium was sterilized by autoclaving, after which the following sterilized components were added: thiamine hydrochloride (4.5 mg/L), glucose (22.1 g/L), trace elements, see Table 12 (10 mL/L), ampilcillin (100 mg/L), and inoculum (seed) (125 mL). The pH was adjusted as needed using ammonium hydroxide (20 vol %) or phosphoric acid (20 vol %). The added components were sterilized either by autoclaving or filtration.

TABLE 11
Composition of Fermentation Medium

| Component | Concentration | |
|---|---|---|
| KH₂PO₄ | 9 | g/L |
| (NH₄)₂HPO₄ | 4 | g/L |
| MgSO₄•7H₂O | 1.2 | g/L |
| Citric Acid | 1.7 | g/L |
| Yeast extract | 5.0 | g/L |
| Mazu DF 204 Antifoam | 0.1 | mL/L |

TABLE 12
Trace Elements

| Component | Concentration, mg/L |
|---|---|
| EDTA | 840 |
| CoCl₂•H₂O | 250 |
| MnCl₂•4H₂O | 1500 |
| CuCl₂•2H₂O | 150 |
| H₃BO₃ | 300 |
| Na₂MoO₄•2H₂O | 250 |
| Zn(CH₃COO)₂•H₂O | 1300 |
| Ferric citrate | 10000 |

The operating conditions for the fermentations are summarized in Table 13. The initial concentration of glucose was 22.1 g/L. When the initial residual glucose was depleted, a pre-scheduled, exponential glucose feed was initiated starting the fed-batch phase of the fermentation run. The glucose feed (see Tables 14 and 15) contained 500 g/L of glucose and was supplemented with 5 g/L of yeast extract. The components of the feed medium were sterilized either by autoclaving or filtration. The goal was to sustain a specific growth rate of 0.13 h⁻¹, assuming a yield coefficient (biomass to glucose) of 0.25 g/g, and to maintain the acetic acid levels in the fermentation vessel at very low values (i.e., less than 0.2 g/L). The glucose feed continued until the end of the run. Induction was initiated with a bolus of 2 g/L of L-arabinose at the selected time (i.e., 15 h of elapsed fermentation time). A bolus to deliver 5 g of yeast extract per liter of fermentation broth was added to the fermentation vessel at the following times: 1 h prior to induction, at induction time, and 1 h after induction time. The fermentation run was terminated after 19.97 h of elapsed fermentation time, and 4.97 h after the induction time.

TABLE 13
Fermentation Operating Conditions

| Condition | Initial | Minimum | Maximum |
|---|---|---|---|
| Stirring | 220 rpm | 220 rpm | 1200 rpm |
| Air Flow | 3 SLPM | 3 SLPM | 30 SLPM |
| Temperature | 37° C. | 37° C. | 37° C. |
| pH | 6.7 | 6.7 | 6.7 |
| Pressure | 0.500 atm | 0.500 atm | 0.500 atm |
| | (50.7 kPa) | (50.7 kPa) | (50.7 kPa) |
| Dissolved O₂* | 20% | 20% | 20% |

*Cascade stirrer, then air flow.

TABLE 14
Composition of Feed Medium

| Component | Concentration | |
|---|---|---|
| MgSO₄•7H₂O | 2.0 | g/L |
| Glucose | 500 | g/L |
| Ampicillin | 150 | mg/L |
| (NH₄)₂HPO₄ | 4 | g/L |
| KH₂PO₄ | 9 | g/L |
| Yeast extract | 5.0 | g/L |
| Trace Elements - Feed (Table 5) | 10 | mL/L |

TABLE 15
Trace Elements - Feed

| Component | Concentration, mg/L |
|---|---|
| EDTA | 1300 |
| CoCl₂•H₂O | 400 |
| MnCl₂•4H₂O | 2350 |
| CuCl₂•2H₂O | 250 |
| H₃BO₃ | 500 |
| Na₂MoO₄•2H₂O | 400 |
| Zn(CH₃COO)₂•H₂O | 1600 |
| Ferric citrate | 4000 |

Isolation and Purification of Peptide:

After completion of the fermentation run, the entire fermentation broth was passed three times through an APV model 2000 Gaulin type homogenizer at 12,000 psi (82,700 kPa). The broth was cooled to below 5° C. prior to each homogenization. The homogenized broth was immediately processed through a Westfalia WhisperFuge™ (Westfalia Separator Inc., Northvale, N.J.) stacked disc centrifuge at 700 mumin and 12,000 RCF to separate inclusion bodies from suspended cell debris and dissolved impurities. The recovered paste was re-suspended at 15 g/L (dry basis) in water and the pH was adjusted to a value between 8.0 and 10.0 using Na₂CO₃/NaOH buffer. The pH was chosen to help remove cell debris from the inclusion bodies without dissolving the inclusion body proteins. The suspension was passed through the APV 2000 Gaulin type homogenizer at 12,000 psi (82,700 kPa) for a single pass to provide rigorous mixing. The homogenized high pH suspension was immediately processed in a Westfalia WhisperFuge™ stacked disc centrifuge at 700 mL/min and 12,000 RCF to separate the washed inclusion bodies from suspended cell debris and dissolved impurities. The recovered paste was resuspended at 15 gm/L (dry basis) in pure water. The suspension was passed through the APV 2000 Gaulin type homogenizer at 12,000 psi (82,700 kPa) for a single pass to provide rigorous washing. The homogenized suspension was immediately processed in a Westfalia WhisperFuge198 stacked disc centrifuge at 700 mL/min and 12,000 RCF to separate the washed inclusion bodies from residual suspended cell debris and NaOH.

The recovered paste was resuspended in pure water at 25 g/L (dry basis) and the pH of the mixture was adjusted to 2.2 using HCl. The acidified suspension was heated to 70° C. for 5 to 14 h to complete cleavage of the DP site separating the fusion peptide from the product peptide without damaging the target peptide. The product slurry was adjusted to pH 5.1 (note: the pH used here may vary depending on the solubility of the peptide being recovered) using NaOH and then was cooled to 5° C. and held for 12 h. The mixture was centrifuged at 9000 RCF for 30 min and the supernatant was decanted. The supematant was then filtered with a 0.45 μm membrane. For some low solubility peptides, multiple washes of the pellet may be required to increase peptide recovery.

The filtered product was collected and concentrated by vacuum evaporation by a factor of 2:1 before lyophilization. Spectrophotometric detection at 220 and 278 nm was used to monitor and track elution of the product peptide.

### Examples 19 and 20

### Hair Coloring Using a Pigment Dispersed with a Methacrylic Acid-Containing Polymeric Dispersant in Conjunction with a Triblock Peptide-Based Conjugate

The purpose of these Examples was to demonstrate the enhanced durability of hair color obtained using a carbon black pigment, dispersed with a methacrylic acid-containing polymeric dispersant, in conjunction with a triblock peptide-based conjugate and to compare it to the durability obtained with the carbon black pigment alone. The peptide conjugate comprised multiple hair-binding peptide, spacer, and PMMA-binding peptide sequences.

The carbon black dispersion was prepared as described in Example 4. The triblock peptide-based conjugate described in Example 18 and given by SEQ ID NO:70 was used. The peptide conjugate (20 mg) was added to 1 mL of deionized water and the mixture was dispersed on a sonicator for 1 min while on ice. The dispersed peptide conjugate was added to a 20 mL scintillation vial containing 40 mg of carbon black dispersion, and 3 mL of deionized water was added to bring the final volume to about 4 mL. The resulting carbon black dispersion was sonicated for 3 min while on ice.

Hair Coloring:

A hair swatch of natural white hair (approximately 1.00 g), obtained from International Hair Importers and Products (Bellerose, N.Y.), was placed in a 20 mL scintillation vial and 8 mL of the carbon black dispersion containing the peptide conjugate was added. The mixture was shaken using an incubator shaker at room temperature for 30 min at 100 rpm to ensure good contact of the pigment dispersion with the hair. The hair swatch was removed from the vial and rinsed with deionized water. After the water rinse, the hair swatch retained most of the black color.

The steps described above were repeated using a carbon black dispersion that did not contain the peptide conjugate. After the water rinse, the color intensity of both hair samples was measured as described in Examples 8-12. The results are given in Table 16.

TABLE 16
Hair Color Intensity after a Water Rinse

| Example | Colorant | Delta E |
|---|---|---|
| 19 | Carbon Black with | 30.2 |
| | Peptide Conjugate | |
| 20, Comparative | Carbon Black | 2.81 |

The results shown in Table 16 demonstrate that the use of the peptide conjugate comprising multiple hair-binding peptide, spacer, and PMMA-binding peptide sequences in conjunction with the methacrylate-coated carbon black pigment resulted in a significant enhancement of the retention of the hair color after a water rinse (Example 19) compared to the use of carbon black alone (Comparative Example 20).

### Example 21

### Selection of Shampoo-Resistant Polymethylmethacrylate (PMMA)-Binding Peptides Using Biopanning

The purpose of this Example was to identify phage peptides that bind to polymethylmethacrylate (PMMA) and are resistant to shampoo washing using a modified phage display biopanning method. After contacting a PMMA substrate with a phage library, the resulting phage peptide-PMMA complexes were washed with a diluted shampoo.

The PMMA material used in the biopanning experiments was polymer resin, Plexiglas VS 100 (about 3 mm diameter beads), from Altuglas International, Arkema Inc., Philadelphia, Pa. The following protocol was used for biopanning against the PMMA beads. Three sets of three PMMA beads were distributed into three tubes, each filled with 1 mL of blocking buffer consisting of 1 mg/mL BSA in TBST containing 0.5% Tween® 20 (TBST-0.5%) and were incubated for 1 h at 4° C. The beads were washed 5 times with TBST-0.5% and then 1 mL of TBST-0.5% containing 1 mg/mL BSA was added to each tube. Then, 10 μL of one of three pooled phage libraries (4×10¹¹ pfu in each tube) was added to the three tubes (i.e., one pooled library per tube containing three PMMA beads) and the beads were incubated for 15 min at 37° C. The three pooled phage libraries consisted of a 1:1 mixture of the following phage peptide libraries (which are described in the General Methods section): the 12-mer linear library (Ph.D.™-12 Phage Display Peptide Library) and the 7-mer linear library (Ph.D.™-7 Phage Display Library), the 15-mer linear library and 20-mer linear library, and the constrained 7-mer library (Ph.D.™-C7C Phage Display Library) and the constrained 14-mer library. The beads were washed 10 times with TBST-0.5%. The beads were then transferred to clean tubes, 1 mL of a non-specific elution buffer consisting of 1 mg/mL BSA in 0.2 M glycine-HCl, pH 2.2, was added to each tube and the tubes were incubated for 10 min. After the incubation, 150 μL of neutralization buffer consisting of 1 M Tris base, pH 9.1 was added to each tube. The eluate from the beads from each tube was used to infect the host cells E. coli ER 2738 (New England BioLabs, Beverly, Mass.), for phage amplifications. The eluate was incubated with an overnight E. coli ER2738 culture diluted 1:100 in LB medium, at 37° C. for 4.5 h. After this time, the cell culture was centrifuged for 30 s and the upper 80% of the supernatant was transferred to a fresh tube, ⅙ volume of PEG/NaCl (20% polyethylene glycol-800, obtained from Sigma Chemical Co. St. Louis, Mo., 2.5 M sodium chloride) was added, and the phage was allowed to precipitate overnight at 4° C. The precipitate was collected by centrifugation at 10,000× g at 4° C. and the resulting pellet was resuspended in 1 mL of TBS. This was the first round of amplified stock.

The amplified first round phage stock was then titered according to the method described below. For the second round of biopanning, more than 2×10¹¹ pfu of phage stock from the first round was used. The biopanning process was repeated as described for the first round. Starting with the third round, shampoo wash steps were added after the phage binding step (i.e., formation of the phage peptide-PMMA complex). Specifically, a shampoo solution, consisting of a 1:1 mix of Neutrogena® Replenishing shampoo and TBS buffer, was used to wash the beads 6 imes with 1 to 2 seconds of vortexing, followed by 6 washes with TBST-0.5%. The beads were then transferred to clean tubes, and the remaining steps were identical to those of the previous round. The fourth or fifth rounds of pannings were done in the same manner as described for the third round.

After the elution step in the final round of biopanning, a small portion (1 to 2 μL) of the eluate from each tube was used to infect 200 μL of mid-log phase bacterial host cells, E. coli ER2738, which were then grown in LB medium for 20 min and then mixed with 3 mL of agarose top (LB medium with 5 mM MgCl₂, and 0.7% agarose) at 45° C. This mixture was spread onto a LB medium/IPTG/ S-Gal™ plate (LB medium with 15 g/L agar, 0.05 g/L IPTG, and 0.04 g/L S-Gal™) and incubated overnight at 37° C. The black plaques were counted to calculate the phage titer. The single black plaques were randomly picked for DNA isolation and sequencing analysis. The amino acid sequences of these shampoo resistant, PMMA-binding phage peptides are given in Table 17.

TABLE 17

Amino Acid Sequences of Shampoo-Resistant
PMMA-Binding Phage Peptides from Pooled Libraries

| | | |
|---|---|---|
| Clone ID | Amino Acid Sequence | SEQ ID NO: |

| | | |
|---|---|---|
| PMMA 1 | APWHLSSQYSGT | 98 |
| PMMA 2 | GLCYRVEPTVCSG | 99 |
| PMMA 3 | HIHPSDNFPHKNRTH | 100 |
| PMMA 4 | HTHHDTHKPWPTDDHRNSSV | 101 |
| PMMA 5 | PEDRPSRTNALHHNAHHHNA | 102 |
| PMMA 6 | TPHNHATTNHHAGKK | 103 |
| PMMA 7 | EMVKDSNQRNTRISS | 104 |
| PMMA 8 | HYSRYNPGPHPL | 105 |
| PMMA 9 | IDTFYMSTMSHS | 106 |
| PMMA 10 | PMKEATHPVPPHKHSETPTA | 107 |
| PMMA 11 | YQTSSPAKQSVG | 108 |
| PMMA 12 | HLPSYQITQTHAQYR | 109 |
| PMMA 13 | TTPKTTYHQSRAPVTAMSEV | 110 |
| PMMA 14 | DRIHHKSHHVTTNHF | 111 |
| PMMA 15 | WAPEKDYMQLMK | 112 |

### Example 22

### Quantitative Characterization of the Binding Affinity of Shampoo-Resistant PMMA-Binding Phage Clones

The purpose of this Example was to quantify the binding affinity of phage clones by titering. Phage clones displaying specific peptides were used for comparing the binding characteristics of different peptide sequences. A titer-based assay was used to quantify the phage binding. This assay measured the output pfu retained by one PMMA bead surface (average of three separate beads). The input for all the phage clones was 10¹² pfu/bead/tube. It should be emphasized that this assay measured the binding of the peptide-expressing phage particle, rather than the isolated peptide binding.

One Plexiglas VS 100 bead was used per tube, and each tube was filled with blocking buffer containing 1 mg/mL BSA in TBST-0.5% and was incubated for 1 h at 4° C. Each bead was washed 5 times with TBST-0.5%. The tubes were then filled with 1 mL of TBST-0.5% containing 1 mg/mL BSA and then purified phage clones (10¹² pfu) were added to each tube. The bead samples were incubated for 15 min at 37° C. and then washed 6 times with shampoo solution, as described in Example 21, followed by 6 washes with TBST-0.5%. Each bead was transferred to a clean tube and 100 μL of a non-specific elution buffer, consisting of 1 mg/mL BSA in 0.2 M Glycine-HCI at pH 2.2, was added. The samples were incubated for 10 min and then 15 μL of neutralization buffer (1 M Tris-HCl, pH 9.2) was added to each tube. The eluted phages from each tube were transferred to a new tube for titering and sequencing analysis.

To titer the bound phages, the eluted phage was diluted with SM buffer to prepare 10-fold serial dilutions of 10¹ to 10⁸. A 10 μL aliquot of each dilution was incubated with 200 μL of mid-log phase E. coli ER2738 (New England BioLabs), and grown in LB medium for 20 min and then mixed with 3 mL of agarose top (LB medium with 5 mM MgCl₂, and 0.7% agarose) at 45° C. This mixture was spread onto a LB medium/IPTG/Xgal plate (LB medium with 15 g/L agar, 0.05 g/L IPTG, and 0.04 g/L Xgal) and incubated overnight at 37° C. The blue plaques were counted to calculate the phage titers, which are given in Table 18 as the average of three determinations made with three separate beads.

TABLE 18
Titer of Shampoo-Resistant PMMA Phage Clones

| Clone ID | SEQ ID NO: | Phage Titer (pfu/bead) |
|---|---|---|
| PMMA 1 | 98 | 8.0 × 10³ |
| PMMA 2 | 99 | 9.1 × 10³ |
| PMMA 3 | 100 | 1.7 × 10⁴ |
| PMMA 4 | 101 | 3 × 10⁵ |
| PMMA 5 | 102 | 1.6 × 10⁴ |
| PMMA 6 | 103 | 9.6 × 10³ |
| PMMA 7 | 104 | 6 × 10³ |
| PMMA 8 | 105 | 2 × 10³ |
| PMMA 9 | 106 | 5 × 10⁵ |
| PMMA 10 | 107 | 4 × 10³ |

The results in Table 18 show that the phage clones bind to PMMA with varying degrees of affinity. SEQ ID NOs:101 and 109 had the highest titers.

What is claimed is:

1. A method for applying a particulate benefit agent to a body surface comprising:
a) providing a particulate benefit agent coated with a polymer;
b) providing a composition comprising a peptide having affinity for the polymer; and
c) applying the coated particulate benefit agent of (a) with the composition of (b) to a body surface for a time sufficient for the coated benefit agent to bind to the body surface.

2. A method according to claim 1 wherein the body surface is selected from the group consisting of hair and skin.

3. A method according to claim 1 wherein the particulate benefit agent is comprised of a material selected from the group consisting of organic pigments, inorganic pigments, metal oxides, metallic nanoparticles, semiconductor nanoparticles, organic nanoparticles, inorganic nanoparticles, and polymer nanoparticles.

4. A method according to claim 3 wherein the particulate benefit agent is comprised of materials selected from the group consisting of D&C Red No. 36, D&C Red No. 30, D&C Orange No. 17, Green 3 Lake, Ext. Yellow 7 Lake, Orange 4 Lake, Red 28 Lake; the calcium lakes of D&C Red Nos. 7, 11, 31 and 34, the barium lake of D&C Red No. 12, the strontium lake D&C Red No. 13, the aluminum lake of FD&C Yellow No. 5, the aluminum lake of FD&C Yellow No. 6, the aluminum lake of FD&C No. 40, the aluminum lake of D&C Red Nos. 21, 22, 27, and 28, the aluminum lake of FD&C Blue No. 1, the aluminum lake of D&C Orange No. 5, the aluminum lake of D&C Yellow No. 10; the zirconium lake of D&C Red No. 33; Cromophthal® Yellow, Sunfast® Magenta, Sunfast® Blue, iron oxides, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide, black titanium dioxide, titanium dioxide nanoparticles, zinc oxide, barium oxide, ultramarine blue, bismuth citrate, hydroxyapatite, zirconium silicate, and carbon black particles.

5. A method according to claim 1 wherein the polymer is selected from the group consisting of polyacrylates, polymethacrylates, polycarbonates, polystyrene, polypropylene, polyethylene terephthalate, polyurethanes, polypeptides, lignin, polysaccharides, polyamides, polyimides, polyaramides, and copolymers comprising at least one monomer from methacylates, acrylates or styrene.

6. A method according to claim 1 wherein the peptide having affinity for the polymer is selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 46, 98, 99, 100, 101, 102, 103, 104, 105, 106, 107, 108, 109, 110, 111, and 112.

7. A method according to claim 1 wherein the body surface is hair and wherein a hair-binding peptide is optionally added to the composition of step (b) comprising a peptide having affinity for the polymer.

8. A method according to claim 1 wherein the body surface is skin and wherein a skin-binding peptide is optionally added to the composition of step (b) comprising a peptide having affinity for the polymer.

9. A method according to claim 1 wherein the peptide having affinity for the polymer is optionally coupled to a peptide having affinity for the body surface.

10. A method according to claim 9 wherein the peptide having affinity for the body surface is coupled to the peptide having affinity for the polymer with a molecular spacer.

11. A method according to claim 9 wherein the peptide having affinity for the body surface is a hair-binding peptide.

12. A method according to claim 9 wherein the peptide having affinity for the body surface is a skin-binding peptide.

13. A method according to claim 7 or 11 wherein the hair-binding peptide is generated combinatorially by a process selected from the group consisting of phage display, yeast display, bacteria display and combinatorial solid phase peptide synthesis.

14. A method according to claim 8 or 12 wherein the skin-binding peptide is generated combinatorially by a process selected from the group consisting of phage display, yeast display, bacteria display and combinatorial solid phase peptide synthesis.

15. A method according to claim 7 or 11 wherein the hair-binding peptide is generated empirically.

16. A method according to claim 8 or 12 wherein the skin-binding peptide is generated empirically.

17. A method according claim 15 wherein the empirically generated hair-binding peptide comprises positively charged amino acids having affinity for hair.

18. A method according claim 16 wherein the empirically generated skin-binding peptide comprises positively charged amino acids having affinity for skin.

19. A method according to claim 7 or 11 wherein the hair-binding peptide is selected from the group consisting of SEQ ID NOs:47, 48, 49, 50, 51, 52, 58, 59, 60, 61, 62, 73, 74, 75, 76, 77, 78, 79, 80, and 81.

20. A method according to claim 8 or 12 wherein the skin-binding peptide is selected from the group consisting of SEQ ID NO:53, 54, 55, 56, 57, 58, 59, 60, 61, 62, 82, 83, 84, 85, 86, 87, 88, 89, 90, 91, 92, and 93.

21. A method according to claim 1 wherein the particulate benefit agent coated with a polymer and the composition comprising a peptide having affinity for the polymer are applied to the body surface concomitantly.

22. A method according to claim 1 wherein the particulate benefit agent coated with a polymer is applied to the body surface prior to the application of the composition comprising a peptide having affinity for the polymer.

23. A method according to claim 1 wherein the composition comprising a peptide having affinity for the polymer is applied to the body surface prior to the application of the particulate benefit agent coated with a polymer.

24. A method according to claim 1 wherein the peptide having affinity for the polymer is generated combinatorially by a process selected from the group consisting of phage display, yeast display, bacteria display and combinatorial solid phase peptide synthesis.

25. A method according to claim 1 further comprising the step of:
d) reapplying the composition comprising a peptide having affinity for the polymer to the body surface.

26. A method according to claim 1 further comprising the step of:
d) applying a composition comprising a polymeric sealant to the body surface.

27. A method according to claim 26 wherein the polymeric sealant is selected from the group consisting of poly(allylamine), acrylates, acrylate copolymers, methacrylates, methacrylate copolymers, polyurethanes, carbomers, methicones, polypeptides, amodimethicones, polyethylenene glycol, beeswax, and siloxanes.

28. A personal care composition comprising:
a) a particulate benefit agent coated with a polymer; and
b) a composition comprising a peptide having affinity for the polymer.

29. A personal care composition according to claim 28 wherein the particulate benefit agent is comprised of a material selected from the group consisting of organic pigments, inorganic pigments, metal oxides, metallic nanoparticles, semiconductor nanoparticles, organic nanoparticles, inorganic nanoparticles, and polymer nanoparticles.

30. A personal care composition according to claim 28 wherein the particulate benefit agent is comprised of materials selected from the group consisting of D&C Red No. 36, D&C Red No. 30, D&C Orange No. 17, Green 3 Lake, Ext. Yellow 7 Lake, Orange 4 Lake, Red 28 Lake; the calcium lakes of D&C Red Nos. 7, 11, 31 and 34, the barium lake of D&C Red No. 12, the strontium lake D&C Red No. 13, the aluminum lake of FD&C Yellow No. 5, the aluminum lake of FD&C Yellow No. 6, the aluminum lake of FD&C No. 40, the aluminum lake of D&C Red Nos. 21, 22, 27, and 28, the aluminum lake of FD&C Blue No. 1, the aluminum lake of D&C Orange No. 5, the aluminum lake of D&C Yellow No. 10; the zirconium lake of D&C Red No. 33; Cromophthal® Yellow, Sunfast® Magenta, Sunfast®) Blue, iron oxides, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide, black titanium dioxide, titanium dioxide nanoparticles, zinc oxide, barium oxide, ultramarine blue, bismuth citrate, hydroxyapatite, zirconium silicate, and carbon black particles.

31. A personal care composition according to claim 28 wherein the polymer is selected from the group consisting of polyacrylates, polymethacrylates, polymethlymethacrylates, polycarbonates, polystyrene, polypropylene, polyethylene terephthalate, polyurethanes, polypeptides, lignin, polysaccharides, polyamides, polyimides, polyaramides, and copolymers comprising at least one monomer from methacylates, acrylates or styrene.

32. A personal care composition according to claim 28 wherein the peptide having affinity for the polymer is selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 46, 98, 99, 100, 101, 102, 103, 104, 105, 106, 107, 108, 109, 110, 111, and 112.

33. A personal care composition according to claim 28 wherein the composition comprising a peptide having affinity for the polymer further comprises a hair-binding peptide.

34. A personal care composition according to claim 28 wherein the composition comprising a peptide having affinity for the polymer further comprises a skin-binding peptide.

35. A personal care composition according to claim 28 wherein the peptide having affinity for the polymer is optionally coupled to a peptide having affinity for a body surface.

36. A personal care composition according to claim 35 wherein the peptide having affinity for the body surface is coupled to the peptide having affinity for the polymer with a molecular spacer.

37. A personal care composition according to claim 35 wherein the peptide having affinity for the body surface is a hair-binding peptide.

38. A personal care composition according to claim 35 wherein the peptide having affinity for the body surface is a skin-binding peptide.

39. A personal care composition according to claim 33 or 37 wherein the hair-binding peptide is generated combinatorially by a process selected from the group consisting of phage display, yeast display, bacteria display and combinatorial solid phase peptide synthesis.

40. A personal care composition according to claim 34 or 38 wherein the skin-binding peptide is generated combinatorially by a process selected from the group consisting of phage display, yeast display, bacteria display and combinatorial solid phase peptide synthesis.

41. A personal care composition according to claim 33 or 37 wherein the hair-binding peptide is generated empirically.

42. A personal care composition according to claim 34 or 38 wherein the skin-binding peptide is generated empirically.

43. A personal care composition according claim 41 wherein the empirically generated hair-binding peptide comprises positively charged amino acids having affinity for hair.

44. A personal care composition according claim 42 wherein the empirically generated skin-binding peptide comprises positively charged amino acids having affinity for skin.

45. A personal care composition according to claim 33 or 37 wherein the hair-binding peptide is selected from the group consisting of SEQ ID NOs:47, 48, 49, 50, 51, 52, 58, 59, 60, 61, 62, 73, 74, 75, 76, 77, 78, 79, 80, and 81.

46. A personal care composition according to claim 34 or 38 wherein the skin-binding peptide is selected from the group consisting of SEQ ID NO:53, 54, 55, 56, 57, 58, 59, 60, 61, 62, 82, 83, 84, 85, 86, 87, 88, 89, 90, 91, 92, and 93.

47. A personal care composition according to claim 28 wherein the peptide having affinity for the polymer is generated combinatorially by a process selected from the group consisting of phage display, yeast display, bacteria display and combinatorial solid phase peptide synthesis.

48. A diblock, peptide-based conjugate having the general structure [(BSBP)ₘ-(PBP)ₙ]ₓ, wherein
a) BSBP is a body surface binding peptide;
b) PBP is a polymer-binding peptide; and
c) m, n, and x independently range from 1 to about 10.

49. A triblock, peptide-based conjugate having the general structure [[(BSBP)ₘ-S_{q}]ₓ-[(PBP)ₙ-Sᵣ]_{z}]_{y}, wherein
a) BSBP is a body surface binding peptide;
b) PBP is a polymer-binding peptide;
c) S is a molecular spacer; and
d) m, n, x and z independently range from 1 to about 10, y is from 1 to about 5, and where q and r are each independently 0 or 1, provided that both r and q may not be 0.

50. A peptide-based conjugate according to claim 48 or 49 wherein the body surface binding peptide is generated combinatorially by a process selected from the group consisting of phage display, yeast display, bacteria display and combinatorial solid phase peptide synthesis.

51. A peptide-based conjugate according to claim 48 or 49 wherein the body surface binding peptide is a hair-binding or skin-binding peptide.

52. A peptide-based conjugate according to claim 51 wherein the hair-binding or skin-binding peptide is generated combinatorially by a process selected from the group consisting of phage display, yeast display, bacteria display and combinatorial solid phase peptide synthesis.

53. A peptide-based conjugate according to claim 51 wherein the hair-binding or skin-binding peptide is generated empirically.

54. A peptide-based conjugate according to claim 53 wherein the empirically generated hair-binding or skin-binding peptide comprises positively charged amino acids.

55. A peptide-based conjugate according to claim 51 wherein the hair-binding peptide is selected from the group consisting of SEQ ID NOs:47, 48, 49, 50, 51, 52, 58, 59, 60, 61, 62, 73, 74, 75, 76, 77, 78, 79, 80, and 81.

56. A peptide-based conjugate according to claim 51 wherein the skin-binding peptide is selected from the group consisting of SEQ ID NO:53, 54, 55, 56, 57, 58, 59, 60, 61, 62, 82, 83, 84, 85, 86, 87, 88, 89, 90, 91, 92, and 93.

57. A peptide-based conjugate according to claim 48 or 49 wherein the polymer-binding peptide is generated combinatorially by a process selected from the group consisting of phage display, yeast display, bacteria display and combinatorial solid phase peptide synthesis.

58. A peptide-based conjugate according to claim 48 or 49 wherein the polymer-binding peptide has affinity for a polymer selected from the group consisting of polyacrylates, polymethacrylates, polymethylmethacrylates, polycarbonates, polystyrene, polypropylene, polyethylene terephthalate, polyurethanes, polypeptides, lignin, polysaccharides, polyamides, polyimides, polyaramides, and copolymers comprising at least one monomer from methacylates, acrylates or styrene.

59. A peptide-based conjugate according to claim 48 or 49 wherein the polymer-binding peptide is selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 98, 99, 100, 101, 102, 103, 104, 105, 106, 107, 108, 109, 110, 111, and 112.

60. A triblock peptide-based conjugate according to claim 49 wherein the spacer is selected from the group consisting of ethanolamine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, ethyl alkyl chains, propyl alkyl chains, hexyl alkyl chains, steryl alkyl chains, cetyl alkyl chains, and palmitoyl alkyl chains.

61. A triblock peptide-based conjugate according to claim 49 wherein the spacer is a peptide comprising from 1 to about 50 amino acids.

62. A triblock peptide-based conjugate according to claim 61 wherein the spacer comprises amino acids selected from the group consisting of proline, lysine, glycine, alanine, serine, and mixtures thereof.

63. A triblock peptide-based conjugate according to claim 61 wherein the spacer comprises peptide sequences selected from the group consisting of SEQ ID NOs:63, 64, 65, 66, 94, 95, 96, and 97.

64. A triblock peptide-based conjugate according to claim 49 wherein the triblock peptide-based conjugate has a sequence selected from the group consisting of SEQ ID NOs:67, 68, 69, and 70.

65. A polymethylmethacrylate-binding peptide selected from the group consisting of SEQ ID NOs: 98, 99, 100, 101, 102, 103, 104, 105, 106, 107, 108, 109, 110, 111, and 112.
